(19) United States
(12) Patent Application Publication
(10) Pub. No.: US 2021/0249132 A1
(43) Pub. Date: Aug. 12, 2021

(54) METHODS AND SYSTEMS FOR TREATING CANCER AND PREDICTING AND OPTIMIZING TREATMENT OUTCOMES IN INDIVIDUAL CANCER PATIENTS

(71) Applicant: Protocol Intelligence, Inc., San Diego, CA (US)

(72) Inventor: Jeffrey A. Colborn, Carlsbad, CA (US)

(21) Appl. No.: 17/152,591

(22) Filed: Jan. 19, 2021

Related U.S. Application Data
(63) Continuation of application No. PCT/US2019/043299, filed on Jul. 24, 2019.
(60) Provisional application No. 62/702,850, filed on Jul. 24, 2018.

(51) Int. Cl. G16H 50/20; G16H 20/00; G06N 20/00; G16H 50/30

(52) U.S. Cl. CPC G16H 50/20; G16H 20/00; G06N 7/005; G16H 50/30; G06N 20/00

(57) ABSTRACT

Methods and systems are provided for predicting and optimizing the outcome of treatment protocols in individual cancer patients including a software training phase and a utilization phase, together including collecting data relating to the characteristics of a patient, determining the treatment and cancer history of the patient, using a mathematical model to infer model parameters associated with the treatments applied to the patient, repeating these steps for multiple patients, employing machine-learning algorithms to search for mathematical relationships between patient characteristics and the model parameters, collecting patient parameters for a new patient, using these data and the machine-learning algorithms to predict model parameters that would apply to the new patient, and using the mathematical model and the model parameters to predict the number of cancer cells versus time and their resistance status in the new patient, along with other aspects of the patient's treatment outcome, under a new proposed treatment plan.

### RELATED APPLICATION DATA

The present application is a continuation of co-pending International Application No. PCT/US2019/043299, filed Jul. 24, 2019, which claims benefit of U.S. provisional application Ser. No. 62/702,850, filed Jul. 24, 2018, the entire disclosures of which are expressly incorporated by reference herein.

### FIELD

The present invention relates to methods and systems for predicting and optimizing the outcome of treatment protocols in individual cancer patients and to methods for treating cancer using such treatment protocols. It relates to personalized cancer treatment, artificial intelligence (AI), machine learning (ML), clinical trial optimization, patient stratification, cancer computational modeling, and utilization of cancer diagnostics.

### BACKGROUND

Cancer is often incurable because it develops resistance to anti-cancer drugs and treatments. Most evidence suggests this is caused primarily, but not necessarily entirely, by random mutation of cancer cell DNA, before and after treatment exposure. Great effort is going into discovering and developing new anti-cancer treatments that may be harder to resist or kill cancer cells more effectively, with fewer side effects. However, as more treatment agents become available, it becomes more important to determine the best protocols with which to use them (in what combinations, order, length of time, dosing) to maximize their effectiveness for each patient and minimize or eliminate treatment resistance. Protocols are currently determined by oncologists using standard of care guidelines, personal experience, and results from clinical trials, which are expensive and time consuming, test just a fraction of the possible treatment schedules, and don't predict results for individual patients. Depending on treatment history, cancer genomics, and other factors, how each agent is used can significantly affect patient survival. As the complexity of this problem grows exponentially with the number of available agents, assistance from decision-support software becomes important for tracking each patient's treatment history, assessing the drug-resistance implications and probable outcomes of various protocols, and optimizing treatment for each patient.

Multiple myeloma is a promising candidate application for such a software tool. For myeloma, there are many potentially effective drugs, resulting in thousands of possible combinations, schedules, and doses. Over fourteen (14) agents are effective against myeloma, with many more in development, yet they encounter resistance in most patients with time. With over 100,000 myeloma patients in the U.S., (0.7%) and total treatment costs of over ten billion dollars per year (7%), myeloma treatment costs ten times the average for other cancers. The disease rate and costs are similar in Europe. Other types of cancer offer further significant needs for protocol optimization, including other hematological cancers such as leukemias and lymphomas, and common solid tumors.

A sometimes important secondary source of resistance is so-called “directed mutation,” which is mutation caused by environmental factors or agents. For example, anti-cancer drugs themselves are known to promote the development of second cancers in some cases. Here, by mutation we mean any change in the structure of a cancer cell's DNA that is hereditable, including point mutations, gene translocations, insertions, deletions, copy number variations, or any other hereditable changes.

A cancer's response and/or resistance to a particular treatment can be affected by other factors as well, such as its expression of RNA and/or proteins, and the tumor microenvironment. Changes in the microenvironment can confer selective advantages to different clones and subclones of the tumor and induce changes in the profile of protein expression by the overall tumor.

Cancer modeling can be categorized into three areas: 1) epidemiological and population-level statistical modeling, 2) mechanistic (including multi-scale) modeling of tumor growth, and 3) modeling of cancer initiation, progression, and drug resistance as the evolution and collective behavior of a population of cancer cells, including subpopulations, clones, and mutants. The first area aims to improve understanding of statistical relationships between patient characteristics and general outcomes. It does not generally attempt to study the effect of specific treatment protocols on specific patients. Results of studies in this area can sometimes be used to suggest candidate hypotheses for predicting relationships between patient data and patient response to treatment.

The second area involves modeling the physical aspects and interactions of tumor material and biological tissues, often in two or three spatial dimensions. This can become important in modeling the evolution, mechanisms, and metastasis of solid tumors; less so with liquid tumors such as myeloma. However, even with liquid tumors of the bone marrow, the interaction between the cancer evolution and the bone marrow microenvironment can be important.

Within the third area of modeling, others have extensively modeled the initiation and clonal evolution of pre-cancerous and cancerous cell populations to better understand tumorigenesis and tumor growth. This has included modeling the order of events, the evolution of driver and passenger mutations, and the role of stem and progenitor cells in cancer initiation.

Others have modeled cancer response to therapy, including immunotherapy and therapies involving viruses. These type of studies can be important for improving understanding of the underlying mechanisms of resistance, but they have not led to development of a practical clinical tool. Rather than modeling the diverse and complex pathways of sequential mutation and clonal expansion, for a practical clinical tool the end result of this process—the birth and growth of each mutant subpopulation (a phenotype) that is resistant to one or more of the treatments employed on the subject patient—is often of more interest. Many researchers have employed this approach to mathematically model cancer drug resistance but have generally not employed ML and have generally only studied simple situations in hypothetical, generic patients. They therefore have not yet been able to account for the diversity of treatments and patient characteristics routinely encountered in the clinic.

The application of AI and ML to cancer detection, prognosis prediction, and treatment planning is a growing field of research and development. Work in this area has included patient survival prediction, cancer classification based on images and other parameters, and treatment planning, including classification and treatment of myeloma and other cancers. However, these approaches generally only provide a list of recommended drugs to test with each individual patient, given his or her specific characteristics. They do not attempt to model and dynamically predict the timing of outcomes of various drug schedules or combinations, which is needed to optimize patient protocols and outcomes. Their usefulness has therefore been limited.

Clinical oncologists currently have myriad protocol options for treating cancers such as myeloma. For each patient, they desire personalized, timely, actionable, and reliable information without a great deal of clinical effort, given the complex and ever-changing sets of available drugs, doses, schedules, and patient cancer genomics, medical conditions, and other characteristics. They would therefore derive great benefit from a decision-support software system that can address this need with features that may for example include dynamically advising which drugs to use for each patient, when to use them, for how long, and in what order, optimized to maximize patient survival under any physician-imposed constraints.

Further, clinical trials have a high failure rate, in part because in many cases they do not demonstrate a significant clinical benefit of the new experimental treatment over the control arm, which is typically the standard of care, in the overall population of patients in the trial. However, in all trials, some patients benefit more than others. If the best-responding patients could be reliably identified in advance, more treatments could be brought to market and could benefit patients. Therefore, developers of new cancer treatments would benefit from a system that could reliably predict which patients will benefit most (response stratification) from new treatments in trial.

### SUMMARY

The present invention relates to methods and systems for predicting and optimizing the outcome of treatment protocols in individual cancer patients in clinical trials and in routine clinical care and to methods for treating cancer using such treatment protocols. In particular, the present invention relates to personalized cancer treatment, artificial intelligence, machine learning, clinical trial optimization, patient stratification, cancer computational modeling, and utilization of cancer diagnostics.

The system comprises computer hardware and software that further comprises a cancer evolver, an artificial-intelligence engine, a database, and a user interface. The method comprises a training phase and a utilization phase, together in one embodiment comprising the steps of collecting data relating to the characteristics of a cancer patient, determining the treatment and cancer history of the patient, using a mathematical model to infer model parameters associated with the treatments applied to the patient, repeating these steps for multiple patients, employing ML or AI algorithms to search for mathematical relationships between the patient characteristics and the model parameters, collecting patient parameters for a new patient, using these data and the ML algorithms to predict model parameters that would apply to the new patient and to treatments thereon, and using the mathematical model and the model parameters predicted for the new patient to predict the number of cancer cells versus time and their resistance status in the new patient, as well as the patient's treatment outcome and other various aspects thereof, under a new proposed treatment plan. The proposed treatment protocol can also be optimized for a desired objective such as maximizing the time before the new patient's cancer reaches a lethal condition.

In one aspect, a method is provided for predicting the outcome of specific treatment protocols on specific cancer patients comprising the following: (a) collecting data at one or more time points relating to the characteristics of a cancer patient, including, for example, the genomics, proteomics, metabolomics, location and size of the tumor, as well as the patient's genomics, demographics, metabolomics, proteomics, and medical condition; (b) determining the treatment history and the number of cancer cells in the patient at multiple times to within some level of approximation via direct measurement, biomarkers, or other means; (c) using a mathematical model of the growth, death, and mutation of cancer cells to infer from step (b) one or more model parameters such as the kill rates and mutation rates associated with the treatments applied to the patient over time; (d) repeating steps (a)-(c) for multiple patients and storing the results in a database; (e) employing one or more ML algorithms or artificial intelligence methodologies to search for mathematical relationships between the data collected in step (a) and the model parameters inferred in step (c); (f) repeating step (a) for a new patient; (g) using the results of steps (e)-(f) to predict model parameters that would apply to the new patient for at least one proposed treatment; (h) using the same or similar mathematical model used in step (c) and the model parameters predicted in step (g) to predict the number of cancer cells versus time and their resistance status in the new patient and various other aspects of the patient's treatment outcome under any proposed treatment for which parameters have been determined in step (g).

In one embodiment, two more steps can be included comprising (i) repeating step (f) for the new patient, which can be done before, during, and/or after steps (g) and (h), and (j) dynamically revising the model parameters predicted in step (g) for the new patient as more measurements become available from step (i). In another embodiment, with or without steps (i) and/or (j), two additional steps can be included comprising (k) specifying constraints on proposed treatments, such as limiting treatment duration for a specific drug to six months or less between three-month rest periods or specifying a three-month holiday from all treatment beginning twelve months after treatment start, and (1) optimizing the planned treatment protocol using a mathematical optimization technique with an objective benefitting the patient, for example, maximizing the time before the new patient's cancer reaches a lethal condition.

The above steps do not necessarily need to be taken in the order given. For example, steps (a) and (b) can be performed concurrently or in reverse order, although it may advantageous if step (a) is started before step (b) is completed. There may be considerable processing of the data from steps (a) and (f) before they are used in steps (e) and other steps. For example, raw genomic tumor data may first be analyzed to determine candidate predictors such as gene expression levels, ratios, gene copy numbers, or type of gene mutation prior to use as predictors in step (e). As another example, patient clinical data may be mathematically combined to form a predictor of kidney function, which may then be used as a predictor input into step (e).

Other aspects and features of the present invention will become apparent from consideration of the following description taken in conjunction with the accompanying drawings.

### BRIEF DESCRIPTION OF THE DRAWINGS

These and other features, aspects, and advantages of the present disclosure will become better understood with regard to the following description, appended claims, and accompanying drawings where:

FIG. 1 is a flow chart of an exemplary method for training a system on patient data using a cancer evolver, an artificial-intelligence engine, a database, and one or more user interfaces.

FIG. 2 is a modeling plot of the log-base10 of the tumor size versus time for the myeloma cancer of Exemplary Patient A. The data points are tumor size measurements derived from blood biomarkers, the shaded regions indicate treatment periods, and the curves are modeled tumor size curves generated by the cancer evolver.

FIG. 3 is a plot of predicted mutation rates versus actual modeled mutation rates resulting from modeling twelve patients undergoing treatment with Revlimid® (lenalidomide) in addition to other drugs. As with Exemplary Patient A, each of these patients refracted from (exhibited resistance to) Revlimid®. In this example, the predicted mutation rates are generated by feeding many patient parameters (predictors) into a support vector machine ML algorithm with squared exponential Gaussian process regression to determine a relationship between the predictors and the mutation rate (response) of each patient for treatment with Revlimid®.

FIG. 4 is similar to FIG. 3 except it applies to the kill rates of the same treatment on the same patients and a support vector machine algorithm with a cubic kernel function was used.

FIG. 5 is a flow chart of an exemplary method for using a system to predict outcomes for various treatment protocols and to optimize such protocols for specific patients using a cancer evolver, an artificial-intelligence engine, a database, and one or more user interfaces.

FIG. 6a is a prediction plot of the log-base10 of the tumor size versus time for the myeloma cancer of Exemplary Patient B. The curves are tumor size curves predicted by the cancer evolver, the shaded regions indicate treatment periods, and the data points are tumor size measurements derived from blood biomarkers. FIG. 6a shows the prediction of the cancer evolution for this patient, and FIG. 6b overlays the actual observed tumor size from biomarkers.

FIGS. 7a-7d show an exemplary model of the cell cycle prior to treatment used in steps (b) and (i) of one embodiment of the method.

FIGS. 8a and 8b show exemplary models of the cell cycle during treatment used in steps (b) and (i) of one embodiment of the method.

FIGS. 9a and 9b are mutation diagrams corresponding to two treatments (FIG. 9a) and four treatments (FIG. 9b) for dividing cancer cells for an example cancer evolution model used in steps (b) and (i) of one embodiment of the method. This example model is based on random mutation and natural selection in the presence of anti-cancer therapy.

FIGS. 10a-10f show a set of “confusion matrices” displaying the true class (the true modeled mutation rate, classified as very low, low, high, or very high corresponding to classes 1, 2, 3, and 4) versus the predicted class for the relapse rates δ for six decision-tree machine learning algorithms. for use in a second exemplary embodiment of the method.

FIGS. 11a and 11b are tables of binned probabilities of the predicted true relapse rate δ for Exemplary Patients C and D for a second exemplary embodiment of the method.

FIG. 12 is a table of binned probabilities of the predicted true number of mutants at start of induction therapy X₁₂ for Exemplary Patients C and D for a second exemplary embodiment of the method.

FIGS. 13a and 13b are histograms of the predicted true relapse rate δ generated by stochastic realizations compared to histograms of the actual modeled relapse rate δ from the independent holdout patients for Exemplary Patients C and D for a second exemplary embodiment of the method.

FIG. 14 shows Synthetic “Kaplan-Meier (SKM) Curves” showing the predicted probability of tumor progression versus time compared to actual Kaplan-Meier curves for the corresponding independent data-sets under proposed RVD induction therapy followed by Revlimid maintenance therapy for a second exemplary embodiment of the method. The SKM curve for Exemplary Patient C is the dashed curve, the SKM curve for Exemplary Patient D is the solid curve, the actual KM curve for Exemplary Patient C is the dotted curve, and the actual KM curve for Exemplary Patient D is the dot-dashed curve.

FIG. 15 is a flowchart showing an exemplary method for predicting the outcome of treatment protocols in cancer patients.

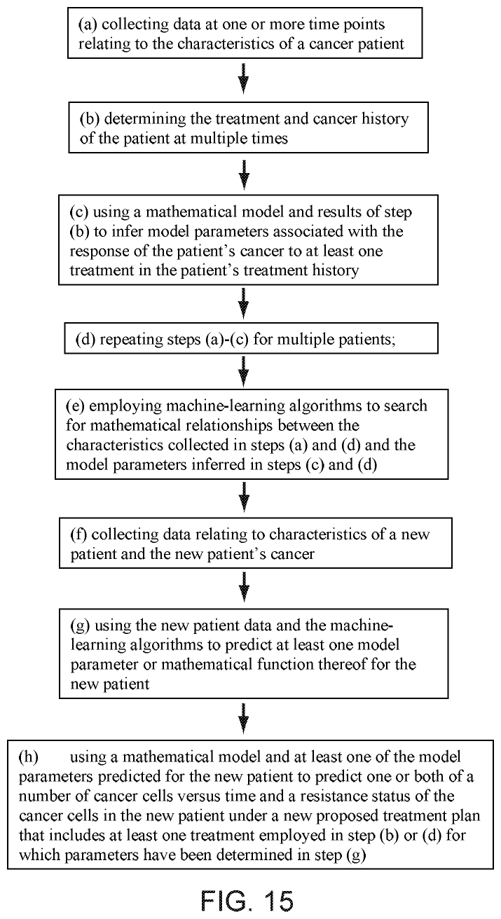

### DETAILED DESCRIPTION OF THE EXEMPLARY EMBODIMENTS

In one aspect, systems and methods of training software on patient data, dynamically predicting the outcomes of specific treatment protocols on specific cancer patients, and methods of optimizing such protocols to maximize the survival of the patient are provided. The provided methods may be applied to any type of cancer, but it is easier to apply in cases for which it is possible to relatively non-invasively measure the tumor size relatively frequently, e.g., via a blood draw or minor surgical procedure biopsy at least once per month or at least once every three months, depending on the growth and kill rates of the cancer without and with treatment respectively, with an accuracy to within a factor of two or at least to within a factor of ten. It is also easier to apply when treating liquid tumors versus solid tumors that may metastasize, because three-dimensional spatial modeling may be simplified or eliminated.

Furthermore, the described methods and system may be more valuable when treating cancers for which there is a large number of potential treatment options, i.e., different drugs, combinations, doses, or schedules that are possible and may be effective. As a counter-example, in the case of a cancer for which there is only one known efficacious drug and only one known effective dose and schedule of administration, the provided method may not be useful. So the described method and system may provide the most value and be most effective for cancers such as myeloma, for which there are over a dozen effective drugs and thousands of potentially effective combinations, dosing, and scheduling options, for which there are available methods to determine the size of the tumor through measurements of bone marrow cells or blood biomarkers, and for which modeling three-dimensional effects is less important than for solid tumors because it is a liquid tumor.

The system to perform the methods and other functionality described herein may include one or more computer systems including one or more processors, memory and/or storage devices, and communication interfaces to provide a cancer evolver, an artificial-intelligence engine, a database, and a user interface. The computer systems may include one or more hardware-based components and/or software-based modules for performing the various functions related to the system, as described elsewhere herein.

In an exemplary embodiment, the system may include one or more electronic devices, e.g., a single computer or network of computer that include one or more processors, memory and/or storage devices, and user interfaces, e.g., a display or other output device and a touch screen, keyboard, mouse, touch pad, and/or other input device, to perform the various functions described herein. Computer programming instructions, e.g., computer programs, software, or firmware, may be stored in the memory, that, when executed, may enable the electronic device to perform one or more of the features described elsewhere herein.

The following description includes steps of exemplary methods for predicting the outcome of treatment protocols in cancer patients, generating new treatment plans for individual patients, and/or for executing treatment plans for individual patients.

First Exemplary Method Embodiment Step (a)

The first step in one exemplary method is to collect data at one or more time points relating to characteristics of a cancer patient, including, for example, the genomics, proteomics, metabolomics, location, and size of the tumor; as well as the patient's genomics, demographics, metabolomics, proteomics, clinical parameters and medical condition. Optionally, a measure or estimate of the error of each measurement may additionally be recorded and used in later steps. In general, any patient characteristics may be collected to test their predictive utility, but it is potentially more effective to select characteristics based on a scientific rationale or theory that suggests their potential usefulness in predicting a particular model parameter that will be inferred and/or needed in later steps of the method.

For example, it is well-established that in most patients a correlation exists between the concentration of a drug in the bloodstream and its effectiveness, within a certain concentration range. In the case of anti-cancer drugs, the expected correlation is between the peak concentration of the drug and/or its persistence in the blood with the rate at which it kills cancer cells. So this implies that it may be useful to collect measurements of each patient's characteristics that generally affect the concentration and/or persistence of drug in the bloodstream. These may include, for example, the drug dose, the patient's height and weight, gender, age, and kidney function estimated from measurements of creatinine and/or glomerular filtration rate.

It also may be important to measure any potential biomarkers that could indicate a specific patient's rate of metabolization of the particular drug being used. It may also be important in this case to measure the size of the tumor as this may also affect the kill rate. It may also be effective to measure the tumor's distribution. For example, the kill rate may be smaller for myeloma tumors that are concentrated in remote areas of bone marrow with relatively poor bloodstream access. It may also be effective to measure biomarkers describing the tumor microenvironment. In addition, the levels of certain proteins in the blood and/or marrow or other tissues may strongly influence the kill rates of certain drugs and so could be used as predictive biomarkers.

For example, the E3 ligase protein cereblon (CRBN) has been identified as a primary target of the immunomodulatory drugs thalidomide, lenalidomide, and pomalidomide, which are important drugs for treating myeloma. Resistance to these drugs has been correlated with a depletion of CRBN in myeloma cells, so measured CRBN levels may help predict kill rates and resistant mutation rates associated with these drugs in a random-mutation mathematical model. A patient's prostate specific antigen (PSA) level has long been thought to correlate with prostrate tumor activity and therefore would be an important characteristic to measure in Step (a) for prostate cancer patients.

A vast array of potentially predictive characteristics is present in the tumor genome and its expression profile. These may be examined at patient diagnosis in step (a) and used in later steps of the process and can additionally be measured at later times. This could include, for example, measurements of the prevalence of various potential driver genetic mutations. For example, the FoundationOne Heme® assay is a next-generation-sequencing (NGS) based assay that identifies genomics alterations in hundreds of cancer-related genes. Activating NRAS mutations predict sensitivity to inhibitors of the MEK and the PI3K/Akt/mTOR pathways, which are pathways commonly exploited by melanomas and some other cancers, so the prevalence of NRAS mutations in the tumor could be measured in Step (a) for patients with these cancers.

As another example, it has been found that the 17p deletion and to a lesser extent the (4;14) translocation aggravate the prognosis of myeloma patients, and other chromosomal abnormalities can indicate a better prognosis. These abnormalities can be measured in a bone marrow sample with fluorescence in-situ hybridization (FISH) and can be expected to be useful in later steps of one or more embodiments of the method.

In general, the whole genome of a patient's tumor may be sequenced, or its exome, and/or its RNA. This can introduce the problem of higher dimensionality to the data, in which there are far more potentially predictive variables in each patient than the number of patients available for training ML algorithms. This problem can be ameliorated using algorithmic techniques or by, for example, requiring a scientific rationale or experimental evidence of a biological mechanism before using a specific genomic mutation or other biomarker as a predictor in the ML algorithms.

### First Exemplary Method Embodiment Step (b)

Step (b) includes determining the treatment history and the tumor size (number of cancer cells) in the patient at multiple times via direct measurement, biomarkers, or other means. Optionally, a measure or estimate of the error of each measurement may additionally be recorded and used in later steps. The treatment history includes the start and stop times for each distinct treatment along with the drugs, dosages, and administration technique comprising the treatment. In an exemplary embodiment, at least two or at least three tumor size measurements are made during each treatment period and significant rest period. In an exemplary embodiment, measurements are made at three or more times from which the size of at least one tumor in the patient or the total amount of cancer in the patient can be inferred.

The achievable accuracy of patient measurements limits the accuracy demands on the model. For example, serum kappa light chain measurements (a myeloma biomarker) have error ranges of 20%-30% due to differences in technique between laboratories, sampling error, patient hydration levels, and other factors. Measurements from bone-marrow biopsies potentially suffer from sampling error, as do biopsies of solid tumors, such that small tumor size measurements may be inaccurate by an order-of-magnitude or more. Significant variation in drug dosage or scheduling may occur due to temporary side effects or administrative causes that may not be captured as model input.

Given these limitations, a reasonable objective may be to model cell populations from patient tumor or biomarker measurements to within an order-of-magnitude or better over time. For the moderate to high growth and kill rates often observed clinically, this generally limits the error in the prediction of event timing due to this error source to within a few weeks to a few months. As the accuracy of diagnostic measurements improves, these error magnitudes decrease. The impact of this uncertainty is ameliorated by the exponential nature of tumor growth and decay, so that a large uncertainty in the tumor size may only correspond to a relatively small change in the predicted timing of the tumor achieving a certain size, for example.

### First Exemplary Method Embodiment Step (c)

Step (c) in one embodiment of the method uses a mathematical model of the growth, death, and mutation of cancer cells to infer from Step (b) one or more model parameters such as the kill rates and mutation rates associated with the treatments applied to the patient over time. Many alternative mathematical models may be used to complete this step, but the model should be based upon known scientific theory. A model may include, e.g., linear and/or non-linear growth dynamics, random and/or directed mutation, spatial effects, and/or interactive effects with normal tissue or the microenvironment. A model may be deterministic or stochastic or contain elements of both. A model may use differential equations to describe phenomena in continuous time or difference equations in discrete time. It can contain varying levels of underlying detail in modeling mechanisms of action of anti-cancer treatments, mechanisms of resistance, clonal composition, evolution and expansion, and cancer stem and progenitor cells and cell differentiation.

Different models may have advantages or disadvantages in their ability to be implemented computationally. For the purposes of the systems and methods herein, it is generally advantageous, but not required, to use the simplest and most computationally efficient model that will meet the requirements of Step (c) and produce sufficiently accurate and reliable results such that use of the method yields overall benefits to patients that outweigh potential harm. Complexities to the model may be added on a case-by-case basis if they improve this accuracy and/or reliability.

An exemplary model that may be used to describe liquid tumors such as myeloma is now described. The model neglects spatial effects. Random mutation is assumed to be the dominant process conferring resistance to each treatment, regardless of the biological mechanism of resistance. Consistent with this assumption, this model distills the complex biological interaction between each treatment and each “subpopulation” into the kill probability per cell cycle, or kill rate. Additionally, there is a probability of mutation of any subpopulation into another subpopulation.

A treatment is defined as one drug, a cocktail of drugs, or any combination of systemic therapies, provided they are concurrent. A subpopulation is defined as a clone or pool of clones that are sensitive or resistant to a specific treatment or set of treatments (a phenotype). Resistance to one treatment is assumed to be generated by a single mutation event. The model allows direct mutation and cross-mutation at independent rates. For example, resistance to two treatments may occur by one mutation event, by two different consecutive independent events, or by all three. The model evolves sensitive, resistant, and cross-resistant subpopulations in discrete time using a single cell cycle as the timestep, which starts simultaneously for all cells immediately after cell division. The model allows for an arbitrary number of differing treatments, which can be applied in any sequence and duration. The model simulates stochastic mutation by numerically evolving deterministic approximations of the stochastic median of each subpopulation. The model includes possible re-birth of resistant subpopulations after they have been eliminated by other treatments. The model allows for full or partial resistance to each treatment to evolve, depending on patient data. The model assumes resistant mutation occurs at a single moment with a single probability, equivalent to identifying it with the final genetic mutation in a potential chain of mutations needed to confer resistance, and any subsequent mutations in this new resistant subpopulation that do not confer resistance to another treatment are neglected. The model includes a (dividing) stem cancer cell and an optional (non-dividing) differentiated cancer cell compartment for each subpopulation. The model limits tumor growth near a maximum allowable tumor size by applying conservation of resources.

For this exemplary embodiment of Step (b), the model includes the following additional assumptions. Cell mutation, division, and death are random Markov processes. The total pre-treatment population and growth rate is dominated by the sensitive subpopulation and is either known from patient data or estimated based on reasonable assumptions and/or data from similar patients. The cancer is born and grows untreated, is then diagnosed, and then treatment begins. All stem cells are assumed to have the same probabilities per cycle of mutation, differentiation and division. All differentiated cells have the same probability of dying. Mutation events resulting in cross-resistance are allowed, but otherwise each resistance mechanism is assumed to be independent. Each cycle is assumed to start simultaneously for all cells immediately after division, which generally does not affect the population dynamics for time frames larger than a cell cycle. However, this last assumption can cause instabilities in the computed cell populations under certain conditions near upper limits imposed by resource constraints. These situations can generally be easily identified and avoided, but if this potential problem is deemed undesirable then a random spread in cycle time can be introduced or an alternative model can be employed based on differential equations rather than difference equations.

Each treatment has a strength, defined by a kill probability, and an activity, defined by a mutation probability, with respect to each subpopulation. Stronger treatments kill more cells at a given concentration, resulting in a higher probability of killing sensitive cells. More active treatments are able to kill a wider variety of mutants, resulting in a lower probability of resistant mutation.

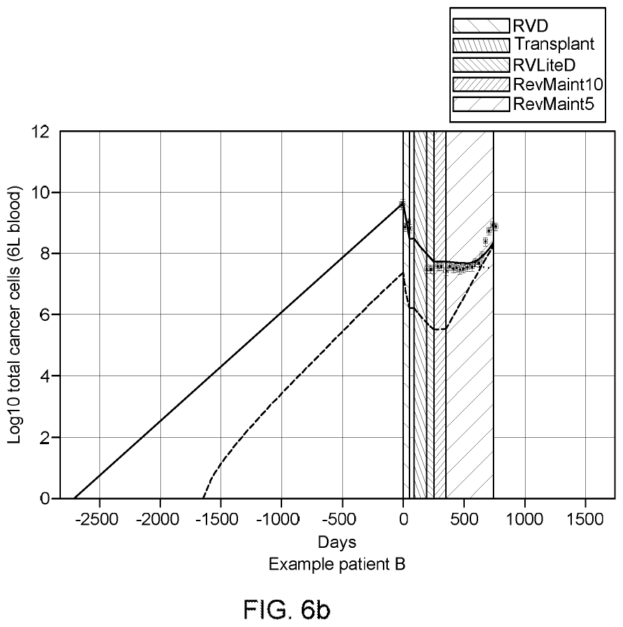

For a single treatment the following notation is used. S is defined as the unmutated population sensitive to the treatment and R as the mutated population resistant to it. Each has a stem compartment and a differentiated compartment given by S^{S}, S^{D}, R^{S}, and R^{D} respectively. This sensitive stem population S^{S} is assumed to be subject to differentiation at the end of the G1 phase in the cell cycle as shown in FIG. 7a and to division just prior to the end of each cycle, after which its value at the end of the n+1 cycle is given by:

S^{S}[n+1]=S^{S}[n]−S_{diff}^{S}[n+1]+S_{div}^{S}[n+1] (1),

where S_{diff}^{S} is the number of sensitive stem cell differentiations, S_{div}^{S} is the number of sensitive stem cell divisions, n is the cycle number, and the effect of mutation on this unmutated population is neglected because the mutation probability is very small, typically of order 10⁻⁵ or less, whereas the differentiation and division probabilities are typically ˜10⁻¹. According to FIG. 7a, the differentiation process is treated first, then division. Equation 1 is then analyzed stochastically, randomly sampling the binomial distribution. [symbol] (N,p) is defined as the integer obtained by randomly sampling the binomial probability distribution N times with probability p and summing.

So, for example, if Nₑᵥₑₙₜ is the number of cells subject as input to a binomial random sampling event and pₑᵥₑₙₜ is the probability of a positive outcome, then [symbol] (Nₑᵥₑₙₜ,pₑᵥₑₙₜ) is the number of cells with a positive outcome after all the cells have been run through the random sampling event once. Each [symbol] (N,p) is a stochastic “realization.” So Equation 1 and FIG. 7a yield:

N_{diff}[n+1]=S^{S}[n] (2a),

N_{div}[n+1]=N_{diff}[n+1]− [symbol] (N_{diff}[n+1],p_{diff}) (2b),

S^{S}[n+1]=N_{div}[n+1]+ [symbol] (N_{div}[n+1],p_{div}) (2c),

where N_{diff} and N_{div} are the number of cells (trials) input into the binomial processes of stem cell differentiation and division for each cycle, respectively, and p_{diff} and p_{div} are the probabilities of a stem cell differentiating and dividing per cycle, respectively. The left-hand sides of Equations 2a-2c define the stochastic cell populations moving clockwise around FIG. 7a and each is input in the right-hand side of the next: N_{diff}[n+1] is the input population for the differentiation process at the end of the G1 phase, N_{div}[n+1] is the input population for the division process at the end of the M phase, and S^{S}[n+1] is the final population at the end of the entire cycle.

Combining Equations 2a-2c gives for each realization:

S^{S}[n+1]=S^{S}[n]− [symbol] (S^{S}[n],p_{diff})+ [symbol] (S^{S}[n]− [symbol] (S^{S}[n],p_{diff}),p_{div}) (3).

The median stochastic solution does not differ appreciably from the analytic solution to the expected value of this equation for populations greater than S^{S}˜10¹-10², which is exceeded soon after the birth of the cancer. An analytic expression can therefore be generated for S^{S} that matches the large values typically measured at diagnosis and can be used as a differentiation source for S^{D} and a mutation source for the resistant stem cell population R. The expected value of equation (3) is taken to obtain:

S^{S}[n+1]=S^{S}[n](1−p_{diff})(1+p_{div}) (4),

which has the solution:

S^{S}[n]=S₀^{S}[(1−p_{diff})(1+p_{div})]ⁿ (5),

and S₀^{S} ≡S[0] is generally assumed to equal one (the cancer starts as a single cell). This gives S^{S}[n]=aⁿ where a∝(1+p_{div})(1−p_{diff})=1+r_{S0}, and r_{S0} is the pre-treatment growth rate of the sensitive stem cells. Now invoking the two-compartment model with stem cells differentiating, then dying, before treatment the sensitive (unmutated) differentiated cell population is given by:

S^{D}[n+1]=S^{D}[n]+S_{diff}^{S}[n+1]−S_{die}^{D}[n+1] (6),

where s_{die}^{D} is the number of sensitive differentiated cell natural deaths. According to FIG. 7b, differentiation is treated first, then natural death, and mutation and division are excluded as these differentiated cells do not mutate or divide. Equation 6 is then analyzed stochastically as before, randomly sampling the binomial distribution. So analogously to Equation 2, Equation 6 and FIG. 7b yield:

N_{diff}[n+1]=S^{S}[n] (7a),

N_{die}[n+1]=S^{D}[n]+ [symbol] B(N_{diff}[n+1],p_{diff}) (7b),

S^{D}[n+1]=N_{die}[n+1]− [symbol] (N_{die}[n+1],p_{die}) (7c),

Combining Equations 7a-7c gives for each stochastic realization:

S^{D}[n+1]=S^{D}[n]+ [symbol] (S^{S}[n],p_{diff})− [symbol] (S^{D}[n]+ [symbol] (S^{S}[n],p_{diff}),p_{die}) (8).

A simulation method is now invoked that allows the use of the analytic solution to the expected value of this equation, with adjustments, to approximate the median stochastic solution. The sensitive stem cells are assumed to differentiate only when the cumulative probability P [n] of a first differentiation exceeds a threshold value close to unity at time n_{diff}. That is, P[n_{diff}]≈1, where:

$$P [ n ] = 1 - {\prod }_{i = 1}^{n} {( 1 - {p}_{diff} )}^{{S}^{S} [ i ]} . \qquad ( 9 )$$

For differentiation (in contrast to mutation), this generally happens very quickly (n_{diff} is generally only a few cell cycles) because the differentiation probability p_{diff}(˜10⁻¹) is much greater than the mutation probability pₘᵤₜₐₜₑ (˜10⁻⁵ or less). The threshold value can be set at, for example, P[n_{diff}]=0.9 in the model.

The expected value of Equation 8 can be taken to get a deterministic equation that can be solved analytically, yielding:

S^{D}[n+1]=(1−p_{die})(S^{D}[n]+p_{diff}S^{S}[n]). (10).

Solving using Z-transforms and assuming S^{D}[n=0]=0 yields the following solution for the differentiated cells for n≥0 pre-treatment:

$${S}^{D} [ n ] = \frac{d {p}_{diff}}{a - d} ( {a}^{n} - {d}^{n} ) . \qquad ( 11 )$$

where d∝1−p_{die} and note that a>1 and d<1 always. Adjusting this solution in accordance with the above simulation method gives the adjusted solution:

$${S}_{A}^{D} [ l ] = ( {S}_{A}^{D} [ l = 0 ] + {S}^{S} [ l = 0 ] \frac{d {p}_{diff}}{a - d} ( {a}^{l} - {d}^{l} ) ) u ( l ) \qquad ( 12 )$$

where l=n−n_{diff}, and u(l) is the unit step function, u(l<0)=0 and u(l≥0)=1. It can generally be assumed that S_{A}^{D}[l=0]=1, that is, the first differentiated cell is born alone. The adjustment entails shifting the solution so it starts the solution with one cell at n_{diff}, which simulates the stochastic median to a very good approximation.

So the solution has a term that grows at the same rate a−1=r_{S0} as the stem cell population and a term that decays at the rate d−1=p_{die}, which is the natural death rate of the differentiated cells. Note this last term in Equations 11 and 12 rapidly decays and becomes negligible for large l. By the time treatment starts, many cell cycles have passed and S_{A}^{D}[l]≅S^{S}[l=0]dp_{diff}a^{l}/(a−d) to an excellent approximation, with the same growth rate as S^{S}[n]=aⁿ. So beyond the initial transient phase, the relative values of p_{diff} and p_{die} will determine the fraction of total cancer cells that are stem cells, and this fraction remains constant unless one or more of these transition probabilities changes.

Turning to the calculation of the resistant mutated stem cells R^{S} without treatment, shown in FIG. 7c,

R^{S}[n+1]=R^{S}[n]+Rₘᵤₜₐₜₑ^{S}[n]−R_{diff}^{S}[n+1]+R_{div}^{S}[n+1] (13),

where now a mutation source exists that adds to the population at the start of each cycle. Employing the probability of mutation per cycle, pₘᵤₜₐₜₑ, gives:

N_{diff}[n+1]=R^{S}[n]+ [symbol] (S^{S}[n],pₘᵤₜₐₜₑ) (14a),

N_{diff}[n+1]=N_{diff}[n+1]− [symbol] (N_{diff}[n+1],p_{diff}) (14b),

R^{S}[n+1]=N_{div}[n+1]+ [symbol] (N_{div}[n+1],p_{div}) (14c),

and so for each stochastic realization:

$${R}^{S} [ n + 1 ] = {R}^{S} [ n ] + ℬ ( {S}^{S} [ n ] , {p}_{mutate} ) - ℬ ( {R}^{S} [ n ] + ℬ ( {S}^{S} [ n ] , {p}_{mutate} ) , {p}_{diff} ) + ℬ ( {R}^{S} [ n ] + ℬ ( {S}^{S} [ n ] , {p}_{mutate} ) - ℬ ( {R}^{S} [ n ] + ℬ ( {S}^{S} [ n ] , {p}_{mutate} ) , {p}_{diff} ) , {p}_{d i v} ) , \qquad ( 15 )$$

where again the stochastic cell populations are defined moving clockwise around FIG. 7c and the resulting equations are combined as for Equation 3. This stochastic equation can be solved numerically. Each solution is a “realization” in time of a possible outcome, given the probabilities.

The above simulation method is again invoked to use the analytic solution to the expected value of this equation to approximate the median stochastic solution. The sensitive cells are assumed to mutate only when the cumulative probability P [n] of a first mutant exceeds a threshold value close to unity at time nₘᵤₜₐₜₑ. That is, P[nₘᵤₜₐₜₑ]≈1, where:

$$P [ n ] = 1 - {\prod }_{i = 1}^{n} {( 1 - {p}_{mutate} )}^{{S}^{S} [ i ]} . \qquad ( 16 )$$

This threshold value P[nₘᵤₜₐₜₑ] can be set, for example to 0.9 in the model. Taking the expected value of equation (15) to obtain a deterministic difference equation for the resistant (mutated) stem cell population before treatment yields:

R^{S}[n+1]=(R^{S}[n]+pₘᵤₜₐₜₑS^{S}[n])(1−p_{diff})(1−p_{div})=(R^{S}[n]+pₘᵤₜₐₜₑaⁿ)a (17),

which may be solved using Z-transforms to give the analytic solution:

R^{S}[n]=pₘᵤₜₐₜₑnaⁿ (18)

before treatment, assuming R[n=0]=0 and S[n=0]=1. Starting this solution with one cell at nₘᵤₜₐₜₑ in accordance with the method of using the analytic solution to the expected value of this equation to approximate the median stochastic solution gives:

R_{A}^{S}[k]=(R_{A}^{S}[k=0]+S^{S}[k=0]pₘᵤₜₐₜₑk)a^{k}u(k) (19),

where R_{A}^{S}[k] is the new, adjusted solution for the resistant stem cells, k=n−nₘᵤₜₐₜₑ, and u(k) is again the unit step function. R_{A}^{S}[k=0]=1 can generally be assumed, that is, the first mutant is born alone.

Turning to the calculation of the resistant mutated differentiated cells R^{D} [n] without treatment shown in FIG. 7d,

R^{D}[n+1]=R^{D}[n]+R_{diff}^{S}[n+1]−R_{die}^{D}[n+1] (20),

where k_{die}^{D} is the number of resistant differentiated cell natural deaths. According to FIG. 7d, differentiation is first treated, then natural death, and mutation and division are excluded as these differentiated cells do not mutate or divide. Equation 20 is then analyzed stochastically as before, randomly sampling the binomial distribution. So analogously to Equations 2 and 3, Equation 20 and FIG. 7d yield:

N_{diff}[n+1]=R^{S}[n] (21a),

N_{die}[n+1]=R^{D}[n]+ [symbol] (N_{diff}[n+1],p_{diff}) (21b),

R^{D}[n+1]=N_{die}[n+1]− [symbol] (N_{die}[n+₁],p_{die}) (21c),

Combining Equations 21a-21c gives:

R^{D}[n+1]=R^{D}[n]+ [symbol] (R^{S}[n],p_{diff})− [symbol] (R^{D}[n]+ [symbol] (R^{S}[n],p_{diff}),p_{die}) (22).

Invoking the simulation method, transforming to k and taking the expected value of equation (22) yields:

R^{D}[k+1]=(1−p_{die})(R^{D}[k]+p_{diff}R_{A}^{S}[k]) (23),

which may be solved analytically using Z-transforms to give:

$${R}^{D} [ k ] = {S}^{S} [ k = 0 ] {p}_{mutate} [ \frac{a {d}^{k + 1}}{{d}^{2} - {a}^{2}} + \frac{d ( k - 1 ) {a}^{k - 2}}{a - d} u ( k - 1 ) ] - {p}_{diff} {d}^{k} u ( k - 1 ) , \qquad ( 24 )$$

and R^{D} [k=0]=0 and R^{S}[k=0]=1 have been used. After the initial transient phase, the middle term dominates and this population grows at the same rate as the unmutated stem cells and R^{D} [k]≅S^{S}[k=0]pₘᵤₜₐₜₑd(k−1)a^{k-2}/(a−d).

The analytic solutions to the equations 4, 10, 17, and 23 have been given assuming they are independent. In practice, these equations may be coupled by resource constraints as the pre-treatment resistance to multiple treatments is tracked, so they must instead be integrated simultaneously through time, while revising the un-treated growth rate a at each time step to accommodate resource constraints. This increases computational requirements but not problematically.

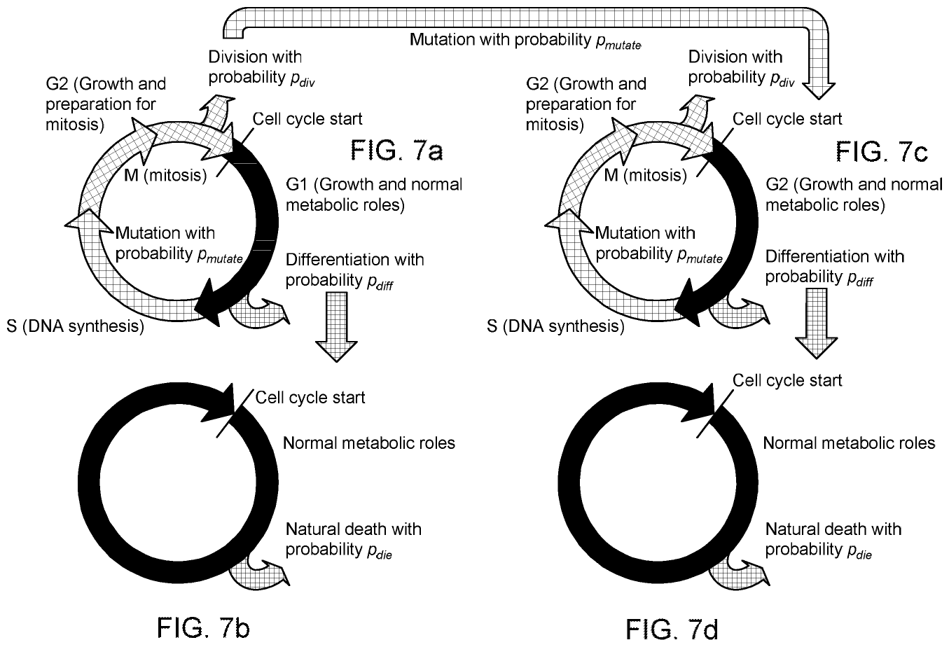

Chemotherapy treatment is now introduced with kill rate pₖᵢₗₗ starting at time n=n* as illustrated in FIGS. 8a and 8b. The resistant cells may be fully resistant or partially resistant. The killing of the cancer cells may occur any time throughout the cell cycle without affecting the results, and this yields the following stochastic equations during treatment for S^{S}, S^{D}, R^{S}, and R^{D}:

S^{S}[n+1]=S^{S}[n]−S_{diff}^{S}[n+1]+S_{div}^{S}[n+1]−Sₖᵢₗₗ^{S}[n+1] (25),

S^{D}[n+1]=S^{D}[n]+S_{diff}^{S}[n+1]−S_{die}^{D}[n+1]−S_{die}^{D}[n+1] (26),

R^{S}[n+1]=R^{S}[n]+Rₘᵤₜₐₜₑ^{S}[n]−R_{diff}^{S}[n+1]+R_{div}^{S}[n+1]−Rₖᵢₗₗ^{S}[n+1] (27),

R^{D}[n+1]=R^{D}[n]+R_{diff}^{S}[n+1]−R_{die}^{D}[n+1]−Rₖᵢₗₗ^{D}[n+1] (28),

Taking the expected values as before gives the following deterministic equations for S^{S}, S^{D}, R^{S}, and R^{D} during treatment:

S^{S}[n+1]=S^{S}[n](1−p_{diff})(1+p_{div})(1−pₖᵢₗₗ^{S}) (29),

S^{D}[n+1]=(1−p_{die})(1−pₖᵢₗₗ^{D})(S^{D}[n]+p_{diff}S^{S}[n]) (30),

R^{S}[n+1]=(R^{S}[n]+pₘᵤₜₐₜₑS^{S}[n])(1−p_{diff})(1+p_{div})(1−pₚₖᵢₗₗ^{S}) (31),

R^{D}[k+1]=(1−p_{die})(1−pₚₖᵢₗₗ^{S})(R^{D}[k]+pₖᵢₗₗR_{A}^{S}[k]) (32),

where pₖᵢₗₗ^{S} is the probability/rate per cell cycle of full kill of the sensitive stem subpopulation, pₖᵢₗₗ^{D} is the probability/rate per cell cycle of full kill of the sensitive differentiated subpopulation, pₚₖᵢₗₗ^{S} is the probability/rate per cell cycle of partial kill of the resistant stem subpopulation, and pₚₖᵢₗₗ^{D} is the probability/rate per cell cycle of partial kill of the resistant differentiated subpopulation. The partial kill rates are zero in the case of full resistance and are fixed in the range 0<pₚₖᵢₗₗ<1 by patient data in the case of partial resistance.

The equations and solutions after treatment stops are of the same form as pre-treatment provided the sensitive cells have not been extinguished by treatment.

The finite size of the human body limits the size of tumors, thus exponential tumor growth cannot continue indefinitely. Many approaches to mathematically modeling this are possible. One approach is to slow the exponential growth rate as the tumor grows using the form C=Cₘₐₓ exp [log(C₀/Cₘₐₓ)exp(−βt)], where β is a fitting parameter, C is the total cancer population, C₀=C(t=0), and Cₘₐₓ is the maximum tumor size as t→∞. Another approach—which is employed in this exemplary model—is to instead assume conservation of mass and other resources consumed by the tumor such that a maximum tumor size Cₘₐₓ is allowed at which there are no remaining resources to fuel tumor growth, and the growth rate of the tumor for C<Cₘₐₓ as C/Cₘₐₓ decreases is simply proportional to the amount of remaining resources until the unconstrained growth rate is reached:

$$r = {r}_{0} ( 1 - \frac{C}{{C}_{max}} ) \qquad ( 33 )$$

where C≤Cₘₐₓ, C and Cₘₐₓ refer to the population of the entire tumor, including stem differentiated, sensitive, and resistant subpopulations, r₀ is the unconstrained growth rate for each subpopulation at each moment in time, and r is the corresponding growth rate constrained by resource limitations according to this model. All subpopulations are assumed to be affected in the same way, and non-cancerous cells are assumed to consume a constant level of resources or consume resources unavailable to the tumor. If the population of a growing tumor exceeds Cₘₐₓ due to cell differentiation, the growth rate becomes negative due to lack of resources, until the total population stabilizes at Cₘₐₓ. Advantages to this approach are there are no free parameters and the constraint is based on simple physical resource limits. This constraint couples the expressions for all of the subpopulations in a feedback loop and therefore requires the equations be numerically integrated over time. The main result is that this resource constraint significantly slows growth only as the tumor grows beyond a few percent of its maximum allowable size.

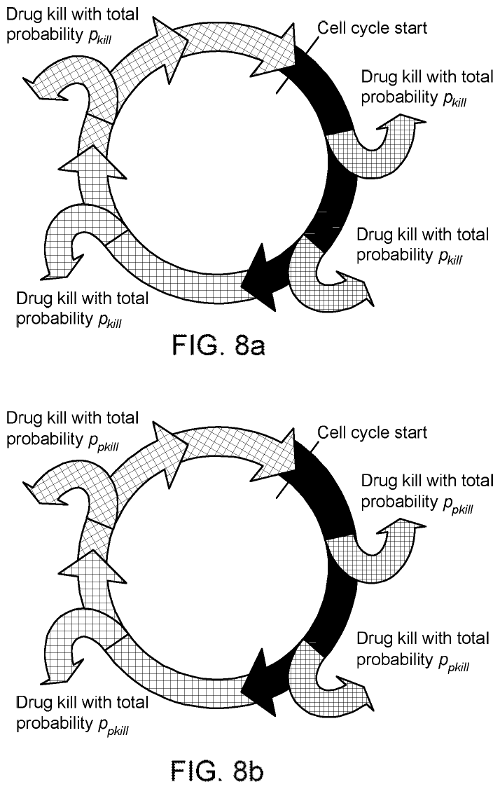

An exemplary model is now described for the situation in which more than one treatment is employed to fight the cancer. FIG. 9a shows the case of two treatments applied to a tumor stem cell subpopulation R₀^{S} (equivalent to S^{S} in the above equations) that is sensitive to both treatments but may or may not be resistant to any other treatments. The subpopulations R₁^{S}, R₂^{S}, and R₁₂^{S} are fully resistant to treatment(s) 1, 2 and both 1 and 2, respectively. These represent only their respective cancer stem cell compartments, as the differentiated cells do not divide or mutate. The mutation rates are shown above each arrow, with pₘ₁ and pₘ₂ the direct mutation rates for resistant subpopulations R₁^{S} and R₂^{S}, respectively, and pₘ₁₂ the cross-mutation rate for the cross-resistant subpopulation R₁₂^{S}. Direct mutation is indicated by solid arrows, and cross-mutation by dashed arrows.

FIG. 9b shows the case of four treatments and illustrates how the number of possible subpopulations and mutation events grows rapidly with increasing number of treatments. N_{subpop}=2^{N}, N is the number of treatments, and N_{subpop} is the number of possible subpopulations. So with two treatments four possible stem subpopulations and three possible mutation processes must be tracked in principle. With four treatments, this grows to sixteen subpopulations and 93 mutation processes, and for seven treatments this becomes 128 subpopulations and 6476 mutation processes. Each time a drug is changed (or replaced, removed, or added to a cocktail) in the clinic, it constitutes a new treatment type, so in a practical situation we may need to model a large number of possible mutant subpopulations and mutation processes. However, many of these subpopulations and mutation processes may be neglected in a simplified model. Employing an analytic simulation of the stochastic median in this type of model is another useful simplification to significantly avoid tracking and interpreting very large numbers of branching stochastic realizations through multiple treatments and possible mutation events.

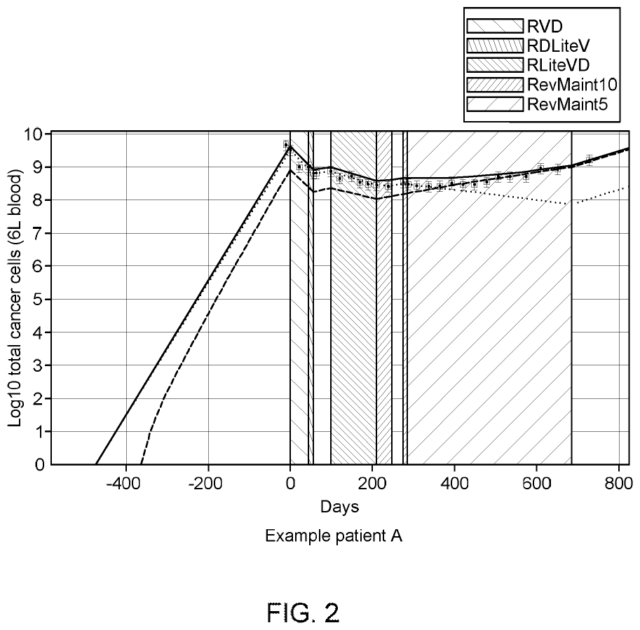

The above example has been implemented in a computer program written in MATLAB® and used to model myeloma patients. FIG. 2 shows the modeling of an Exemplary Patient A, a myeloma patient who has undergone treatment with a cocktail of Revlimid®, Velcade®, and dexamethasone (RVD), followed by RVD with a lighter dose of Velcade® (RDLiteV), followed by a treatment break, followed by RDLiteV with a lighter dose of dexamethasone (RLiteVD), followed by 10 mg/day of Revlimid® (RMaint10), followed by 5 mg/day of Revlimid® (RMaint5). The RVD treatment was given using a 21-day cycle with 25 mg/day of Revlimid® (lenalidomide) given on days 1-14, 1.3 mg/m² Velcade® (bortezomib) given on days 1,4,8, and 11, and 20 mg of dexamethasone given on days 1, 2, 4, 5, 8, 9, 11, 12. The RDLiteV treatment was the same as RVD except the dose of Velcade® was lowered to 1.0 mg/m². The RLiteVD treatment was the same as RDLiteV except the dose of dexamethasone was lowered to 10 mg.

The model parameters were adjusted to fit the patient data as shown in FIG. 2. The estimated pre-treatment growth rate (cancer cell division probability) was 0.0471. The inferred net kill rates for the various treatments on the sensitive cancer cell populations resulting from this modeling run were 0.0285, 0.0350, 0.0086, 0.0004, and 0.0035 for RVD, RDLiteV, RLiteVD, RMaint10, and RMaint5, respectively. The inferred kill rate for RMaint5 on the cancer cells resistant to it was −0.0042. The fact that this inferred kill rate was negative indicates the growth rate of this resistant subpopulation is slightly greater than the estimated untreated growth rate during that time, most likely due to error/uncertainty in this estimate.

Because this model assumes a cell cycle of one day, these kill rates are per day, equivalent to a daily kill probability per cancer cell. The only relevant (i.e., required to match the modeled data) mutation rate for this patient is the rate of mutation from the sensitive population into the population resistant to Revlimid® as a single agent. This is because the only observed resistance is against RMaint5 toward the end of treatment. The model produced the best fit to the data with a value of 6.55×10⁻⁴ for this mutation rate. This produced the inferred resistant subpopulation shown as the dashed curve in FIG. 2.

First Exemplary Method Embodiment Step (d)

Steps (a)-(c) above are repeated for a number of patients in a sample of patients undergoing similar treatments to Exemplary Patient A. Note that the duration of the treatments need not be identical in every patient in order to complete this step. The mutation rates and kill rates inferred by the model in Step (c) of this example embodiment are independent of the duration of the various treatments. These mutation and kill rates were then stored in an Excel® file along with other patient characteristics determined in Step (a). However, these data may be stored in any other type of file or database.

First Exemplary Method Embodiment Step (e)

In this example, the data in the Excel® file created in Step (d) was then fed into the MATLAB® Regression Learner and 19 different ML algorithms were tested on the data and two figures of merit were compared—the root-mean-squared-error (RMSE) and the coefficient of determination (R²) for the predicted responses versus the actual responses. For this example, the predictors tested against the mutation rate into Revlimid® resistance as response were the initial kill rate of RVD plus the following chromosomal abnormalities of the myeloma cancer cells in each patient: the presence of a 17p deletion, the (11:14) translocation, and the overall number of chromosomal deletions, trisomies or monosomies. These genomic predictors were obtained using FISH in Step (a) using standard techniques with commercially available equipment.

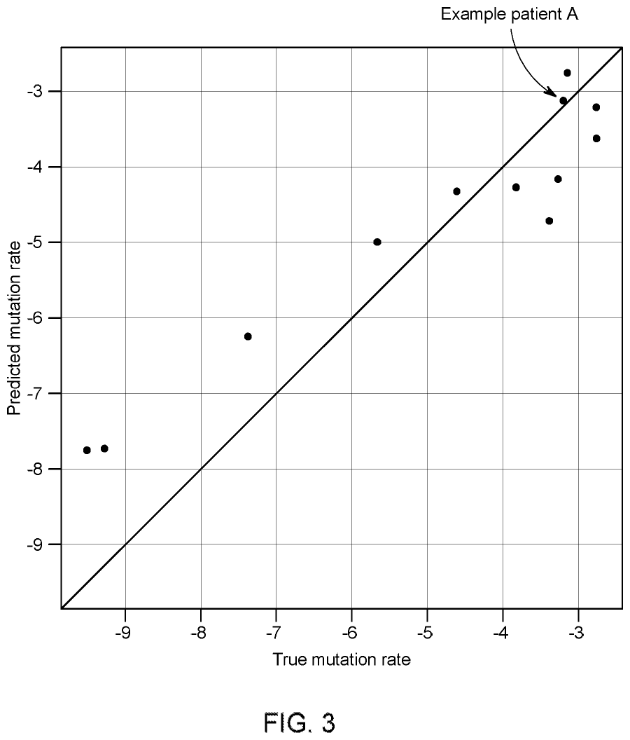

The log₁₀ of the predicted mutation rate versus the actual mutation rate are shown in FIG. 3 for twelve patients using 5-fold cross-validation, i.e., one fifth of the patients were randomly withheld from training the ML algorithms and then used for testing the algorithms, and this process was repeated four times. In this example, this yielded an RMSE=0.94 and R²=0.84. The data point for Exemplary Patient A modeled in Step (b) of this example is indicated by the arrow in FIG. 3. These results were generated using the squared exponential Gaussian process regression algorithm available on the MATLAB® Regression Learner application, which produced the best results from nineteen competing ML algorithms for this particular data set. Similarly, the nineteen algorithms were run in competition on their ability to predict the net kill rates of the various treatments used on this and other patients.

The net kill rate is defined as pₖₙ=1−(1−pₖ)(1+rᵤ), where pₖ is the kill rate and rᵤ is the untreated growth rate. The net kill rate is used in this step of the process because it is directly tied to measurements of the tumor size during treatment. In this example, the predictors tested against the net kill rates of the various treatments as response were the patient gender, age, height, weight, body surface area, body mass index, calculated glomerular filtration rate, measured creatinine, and measured peak pre-treatment light-chain serum concentration. For all but the first treatment (RVD), the RVD kill rate was also used as a predictor, which improved results. These predictors were obtained in Step (a) from routine clinical patient measurements.

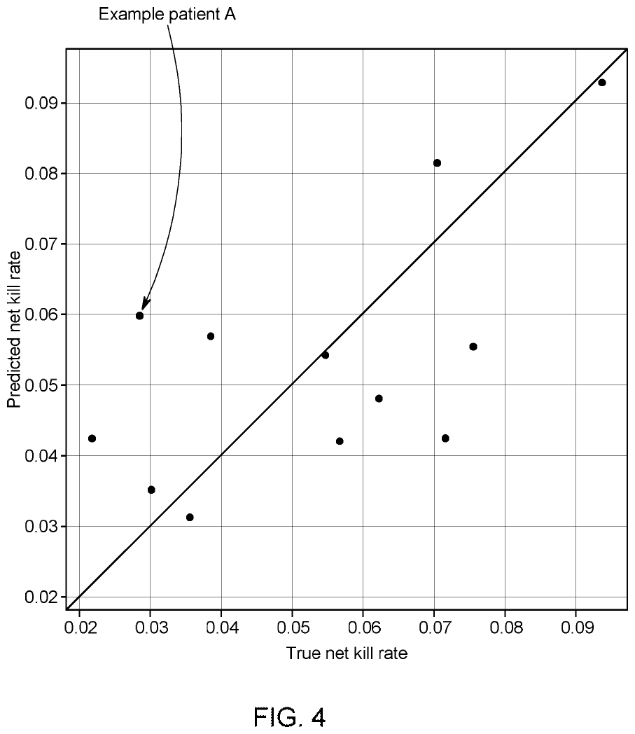

The predicted RVD kill rate versus the actual kill rate is plotted in FIG. 4 for twelve patients, resulting in an RMSE=0.02 and R²=0.27 from a support vector machine (SVM) algorithm with a quadratic kernel function, which produced the best results out of the eighteen competing algorithms. The data point for Exemplary Patient A modeled in Step (b) of this example is indicated by the arrow in FIG. 4. This process was repeated with many patients, including patients that underwent different or additional treatments to those of Exemplary Patient A, and ML algorithms were generated to predict the mutation rates and kill rates of multiple treatments, including some used on Exemplary Patient A and some not used on Exemplary Patient A.

First Exemplary Method Embodiment Step (f)

In Step (f), Step (a) is repeated for a new patient. In the present example, data fields were collected on a patient, Exemplary Patient B, who underwent a protocol with some of the same treatments as Exemplary Patient A. The data fields (patient characteristics) collected on Exemplary Patient B were the same as on Exemplary Patient A. Exemplary Patient B was not part of the patient set used to train the algorithms in Step (e).

First Exemplary Method Embodiment Step (g)

In Step (g), the results of Steps (e) and (f) are used to predict model parameters that would apply to the new patient for at least one proposed treatment plan. In this example, the ML algorithms generated in Step (e) are used with patient characteristics (input predictors) found in Step (f) on Exemplary Patient B to predict the kill rates and mutation rates for various treatments proposed for Exemplary Patient B.

All these proposed treatments need not have been used in any one patient used in Steps (a)-(c), but there must have been at least one patient used in Steps (a)-(c) who underwent each treatment proposed for the new patient being considered in this Step (g). To illustrate this, a set of treatments on Exemplary Patient B is chosen that differs from those on Exemplary Patient A. The treatment plan proposed in this Step (g) for Exemplary Patient B includes RVD, followed by melphalan with an autologous stem cell transplant, followed by RVLiteD, followed by RevMaint10, then RevMaint5. The treatment protocol for RVLiteD is the same as RVD but the dose of dexamethasone is lowered to 10 mg. The melphalan autologous stem cell transplant includes a total melphalan dose of 200 mg/m² of body surface area over days 1 and 2, followed by a transplant of the patient's own stem cells. The resulting predicted mutation rate for Revlimid® and kill rates for RVD, Transplant, RVLiteD, RevMaint10, and RevMaint5 were stored in and Excel® file. These predicted net kill rates for the various treatments on the sensitive cancer cell populations resulting from this modeling run were 0.0471, 0.0028, 0.0091, −0.0021, and 0.0014 for RVD, Transplant, RVLiteD, RMaint10, and RMaint5, respectively. The predicted kill rate for RMaint5 on the cancer cells resistant to it was −0.0163. The predicted mutation rate for resistance to RMaint5 was 3.027e-6 for this modeling example.

First Exemplary Method Embodiment Step (h)

In this Step (h), the same or a similar mathematical model is used as was used in Step (c) and the model parameters predicted in Step (g) is used to predict the number of cancer cells versus time and their resistance status in the new patient (and hence the patient's treatment outcome) under any proposed treatment for which parameters have been determined in Step (g). The resistance status of a cancer cell may be sensitive, partially resistant, or fully resistant to each proposed treatment.

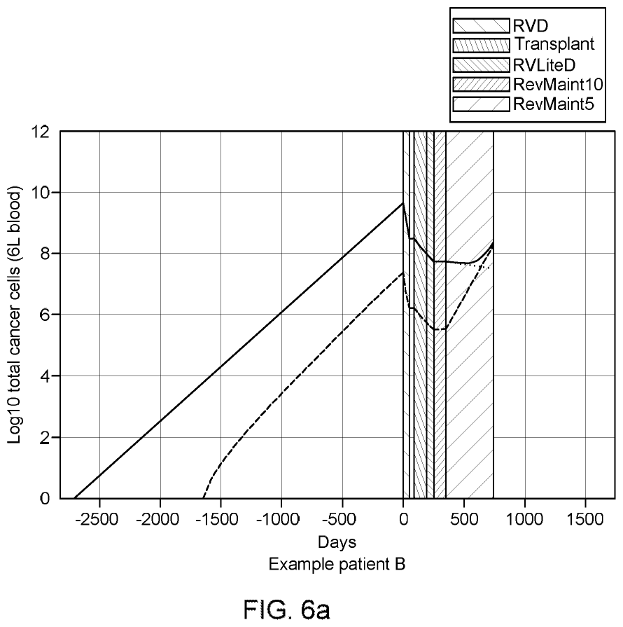

For Exemplary Patient B, the proposed treatment plan is shown in FIG. 6a along with the predicted tumor size versus time (patient outcome) for this proposed treatment plan. Note that while the individual treatments (e.g., drugs or drug combinations) must be chosen from those processed in Step (g) in order to predict the model parameters as responses to the ML algorithms, the timing, duration, and (in this example embodiment) the order of the individual proposed treatments do not have to be chosen until this Step (h). Any arbitrary timing or duration can be proposed for the treatments selected for the treatment plan for Example Patient B and modeled in this example embodiment of Step (h).

The treatment plan proposed in Step (g) and modeled in Step (h) was applied to Exemplary Patient B. FIG. 6b shows the actual subsequent measurements of tumor biomarkers for Exemplary Patient B overlaid on the predictions generated in this Step (h) from FIG. 6a, giving an illustration of the predictive accuracy for this example patient under this example embodiment.

First Exemplary Method Embodiment Step (i)

In optional Step (i), Step (f) is continuously or repeatedly performed for the new patient, which can be done before, during, and/or after Steps (g) and (h). This provides updated measurements of tumor size biomarkers for the new patient, which can be used to revise and improve the predictions of the tumor evolution and outcome for this patient under the proposed treatment plan or any alternative treatment plan in Step (j).

First Exemplary Method Embodiment Step (j)

In this optional Step (j), in one embodiment the model parameters predicted in Step (g) for the new patient are dynamically revised as more measurements become available from Step (i), with the aim of improving the accuracy and reliability of the predictions of the tumor evolution under the currently selected or any alternative treatment plan. As additional treatments are applied to this patient, their kill rates and other performance parameters can also be used as additional predictors and fed into the AI Engine in Step (g) again to predict the kill rates and other parameters for other future planned treatments.

First Exemplary Method Embodiment Step (k)

In this optional Step (k), constraints are specified on proposed treatments or on the overall proposed plan. These can be for example motivated by trying to limit side-effects, accommodate patient lifestyle, financial constraints, or for any other reason. Examples include limiting treatment duration for a specific drug to 6 months or less between 3-month rest periods, specifying a 3-month holiday from all treatment beginning 12 months after treatment start, limiting treatment options to treatments for which data are available from Step (d) exceeding a specified quality or quantity threshold, or excluding a specific drug or drug combination from consideration.

First Exemplary Method Embodiment Step (l)

In this optional Step (l), the planned treatment protocol is optimized under the constraints imposed in Step (k), or under no constraints, using a mathematical optimization technique with an objective benefitting the patient. Example objectives include maximizing the time before the new patient's cancer reaches a lethal condition, maximizing the time before the patient's cancer begins to progress or grow beyond a certain size, probability of cure, minimization of the number of resistant subpopulations, minimization of the number of treatments to yield no progression in a specified timeframe, or any other objective that can be derived from the predictions of Step (h) and/or Step (j).

In a second exemplary method embodiment, a classification ML technique is used, and the cancer is modeled as including two subpopulations of cells subject to exponential growth and decay, one sensitive to treatment and another resistant to treatment. The results of several ML algorithms are pooled to generate a probability distribution of the time to progression for patients of each class. This is further described as follows.

Second Exemplary Method Embodiment Step (a)

The first step in this exemplary embodiment is to collect data at one or more time points relating to the characteristics of a cancer patient, in a manner similar to that of the first exemplary method embodiment. Optionally, a measure or estimate of the error of each measurement may additionally be recorded and used in later steps.

Second Exemplary Method Embodiment Step (b)

In this embodiment, Step (b) comprises determining the treatment history and the tumor size (number of cancer cells) in the patient at multiple times via direct measurement, biomarkers, or other means, in a manner similar to that of the first exemplary method embodiment. Optionally, a measure or estimate of the error of each measurement may additionally be recorded and used in later steps.

Second Exemplary Method Embodiment Step (c)

Step (c) in this embodiment of the method uses a mathematical model of the growth, death, and mutation of cancer cells to infer from Step (b) one or more model parameters such as the kill rates and mutation rates associated with the treatments applied to the patient over time. Many alternative mathematical models may be used to complete this step, but in this embodiment an exponential fit is used with fully resistant mutants, and it is applied to a patient population undergoing a two-treatment protocol comprising induction therapy and maintenance therapy.

This model is simpler than the model used in the first exemplary embodiments; it assumes the existence of two types of cells. The wild type cells are susceptible to all treatments, and the mutant cells are assumed to be resistant to all treatments. Therefore, the dynamics of the total number of cells, Z(t), can be expressed as follows:

Z(t)=X₀*e^{-γ(t-t}^{₁}⁾+X₁₂*e^{δ(t-t}^{₁}⁾ (34)

where t₁ is the time of start of induction treatment, X₀* is the number of wild-type susceptible cells (resistant to zero treatments) at time t₁, X₁₂* is the number of mutant cells (resistant to treatments 1 and 2) at time t₁, γ is the decay rate of the susceptible cell population during treatment and δ is the growth rate of the mutant cell population during treatment. This model is applied only to times t≥t₁. X₁₂* is related to the mutation rate of sensitive cells to resistant cells and to the age of the tumor at time t₁. The time to progression t_{P} is defined mathematically for this example embodiment as the time after start of treatment when the tumor goes from shrinking to growing, i.e., when the two terms on the right-hand side of Equation 34 are equal:

$${t}_{P} = {t}_{1} + \frac{ln ( {X}_{0}^{*} / {X}_{12}^{*} )}{δ + γ} . \qquad ( 35 )$$

Second Exemplary Method Embodiment Step (d)

In this embodiment, Steps (a)-(c) above were repeated for a number of patients in a sample of patients undergoing similar treatments. For this embodiment, the induction treatment (treatment 1) was a combination of Revlimid® (lenalidomide), Velcade® (bortezomib), and dexamethasone (RVD), and the maintenance treatment (treatment 2) was 10 mg/day of Revlimid®. The RVD treatment was given using a 21-day cycle with 25 mg/day of Revlimid® on days 1-14, 1.3 mg/m² Velcade® on days 1, 4, 8, and 11, and 20 mg of dexamethasone on days 1, 2, 4, 5, 8, 9, 11, 12. Some variation in dose and schedule is acceptable between patients, and this can result from missed appointments, varying tolerances to side effects (e.g., requiring temporary dose reductions for some patients), and other factors.

The model parameters and the time to progression inferred by the model in Step (c) of this example embodiment for each patient were stored in an Excel® file along with other patient characteristics determined in Step (a). However, these data may be stored in any other type of file or database. In this example, 56 patients were modeled, and all of these were observed to progress within the time period of observation and therefore it was possible to infer a time to progression for these patients. Of these, 45 were randomly selected for training algorithms in step (e) and the remaining eleven were set aside as an independent test set.

Second Exemplary Method Embodiment Step (e)

In this example embodiment, the data in the Excel® file created in Step (d) was then fed into the MATLAB® Classification Learner and 6 different decision-tree ML algorithms were trained on the training dataset of 45 patients. These decision-tree algorithms used the following approaches: complex tree (maximum of 100 splits), medium tree (maximum 20 splits), simple tree (maximum 4 splits), boosted tree ensemble (AdaBoost with 30 learners), bagged tree ensemble (30 learners), and randomly under-sampled boosted tree ensemble (30 learners). The ML algorithm response used in this case was log₁₀ δ, the rate of progression in the model.

However, other model parameters such as X₁₂* may be used, and many other choices are possible, depending on the model employed in step (c).

An important distinction of this embodiment is that the ML algorithm response chosen must be a parameter of the model chosen in step (c) or a mathematical function of one or more such parameters. The ML response may be classified by any convenient method into any number of classes. In this example, the responses were classified into the following four ranges based upon the distribution of their values: log₁₀ δ<−6, −6≤log₁₀ δ<−3.2, −3.2≤log₁₀ δ<−2.55, −2.55<log₁₀ δ, which are labeled as very low, low, high, and very high, corresponding to class labels 1, 2, 3, and 4.

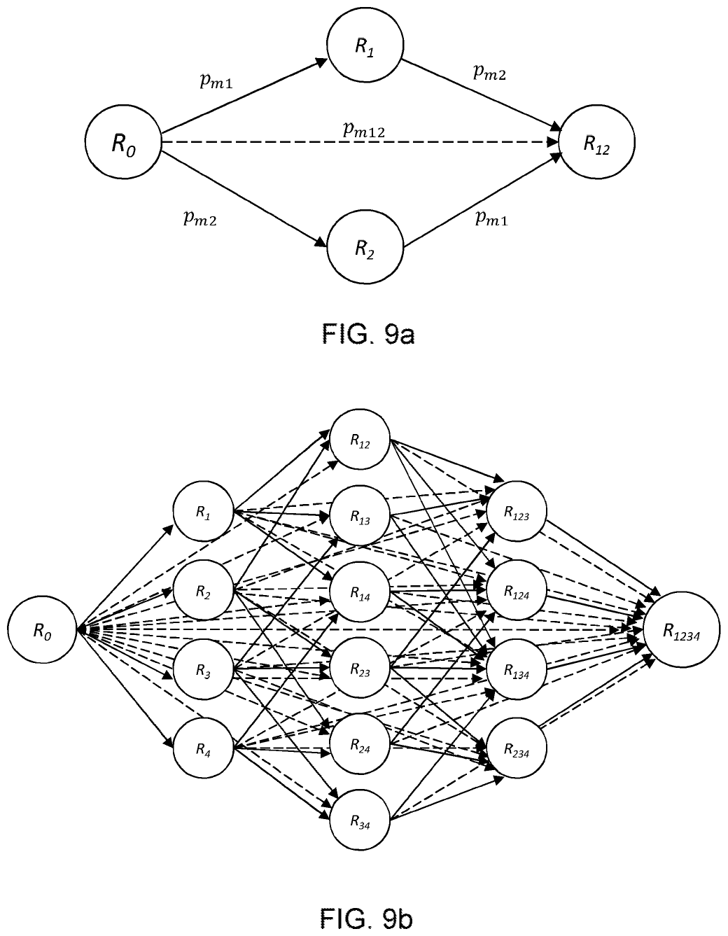

These six algorithms were trained on the following predictors and the percentages of overall classification accuracy were compared and tabulated in confusion matrices, as shown in FIGS. 10a-10f. Each confusion matrix in FIG. 10 shows the true class of log₁₀ δ versus its predicted class 1, 2, 3, and 4. For this example, the predictors used were each patient's age, gender, race, height, weight, serum creatinine, body-mass index (BMI), log₁₀ of the maximum free light chain blood serum concentration prior to start of treatment, the glomerular filtration rate (calculated using the CKD formula), and the following chromosomal abnormalities and biomarkers of the myeloma cancer cells in each patient: the presence of a 17p deletion, the (4:14) translocation, the (11:14) translocation, and the presence of any other chromosomal amplifications, deletions, or translocations. These genomic predictors were obtained using FISH in Step (a) using standard techniques with commercially available equipment. It can be noted from FIGS. 10a-10f that the algorithms have different patterns of prediction and overall accuracies for this particular dataset. The resulting average overall classification accuracy for the six decision-tree algorithms was 33%, modestly but significantly higher than the expected random overall classification accuracy of 25% for this case. Perfect accuracy of the ML algorithm would result in numbers appearing only on the diagonal of the matrix from upper left to lower right, and perfectly random prediction would result in approximately the same numbers appearing in every position of the matrix. The ML algorithms were run using 5-fold cross-validation, i.e., one fifth of the patients were randomly withheld from training the ML algorithms and then used for testing the algorithms, and this process was repeated four times. The six trained algorithms were then stored in computer memory for use in later steps of the exemplary method.

Second Exemplary Method Embodiment Step (f)

In this embodiment of Step (f), Step (a) is repeated for a new patient. In the present example, data fields were collected on Example Patients C and D, selected from the independent test set population of eleven patients. The data fields (patient characteristics) collected on Exemplary Patients C and D were the same as on the training data set of 45 patients modeled in Step (c).

Second Exemplary Method Embodiment Step (g)

In Step (g), the results of steps (e) and (f) are used to predict model parameters that would apply to the new patient for at least one proposed treatment plan. In this example, the six decision-tree ML algorithms generated and selected in Step (e) were used with patient characteristics (input predictors) found in Step (f) on Exemplary Patients C and D to predict the model parameters δ and X₁₂* for the same treatment protocols applied to these Exemplary Patients C and D. In this example, the following statistical technique is used to generate a number of stochastic predictions of the model parameters, which are then used to make a number of stochastic predictions (realizations) of the time to progression of the tumors in Exemplary Patients C and D. To do this, probability distributions are created of the algorithm used, the predicted class of the model parameter δ, and predicted actual class of the model parameter δ, and then used to generate “synthetic Kaplan-Meier” (SKM) curves for the probability versus time for the time to progression for Exemplary Patients C and D.

To start, results of the six decision-tree algorithms are pooled as follows, which yields more stable predictions than relying on only one of the algorithms. First, weights are assigned to each of the six algorithms, depending on the emphasis desired for each. For example, if it is desired to rely more heavily on tree ensemble algorithms, these algorithms can be weighted more heavily. For this example, equal weights are assigned to the six algorithms. A probability distribution function is then created and randomly sampled to yield each stochastic realization. All six of the decision-tree algorithms used in Step (e) are run on the patients in the holdout data set of 11 patients and Exemplary Patients C and D are selected therefrom and a response is determined for each, i.e., the resistant growth rate δ is predicted to be very low, low, high, or very high (classes 1 through 4). This determines the column in FIGS. 10a-10f in which Exemplary Patients C and D fall. In this example, the algorithms predicted resistant growth rate classes of 4, 4, 4, 2, 4, 4 for Exemplary Patient C and 1, 1, 1, 2, 2, 3 for Exemplary Patient D for each of the six algorithms shown in FIGS. 10a-10f, respectively.

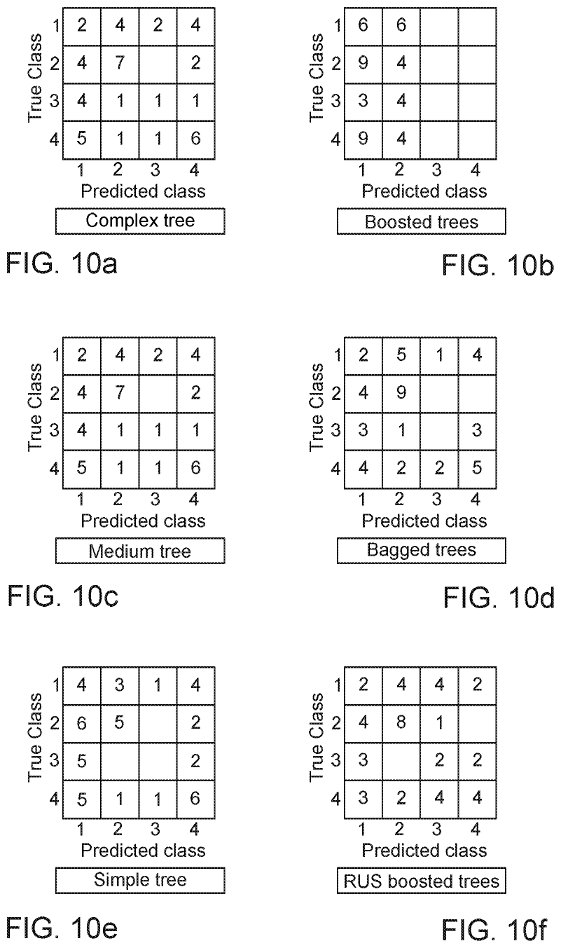

These determine the columns in the confusion matrices in FIGS. 10a-10f that are used to construct a binned probability distribution of the prediction of the true resistant growth rates for Exemplary Patients C and D. By this approach, the predictive accuracy of the algorithms may be improved by accounting for the various probabilities of their misclassification of the responses. In this example, a binned probability distribution is constructed of the true relapse rate δ for this patient from the values in the appropriate columns. Using this approach, a probability may be assigned of the predicted true relapse rate δ for Exemplary Patient C being of each class as shown in FIG. 11a and for Exemplary Patient D in FIG. 11b. Note, for example, that given the prediction by the complex tree algorithm that the class of the relapse rate δ is very high (column 4 in FIG. 10a), the predicted true class is expected to fall within a binned probability distribution given by the numbers in the fourth column of FIG. 10a divided by the total of this column, so these are the probabilities shown in FIG. 11a for the complex tree algorithm.

Note for this example that even though the six algorithms favor predicting a very high relapse rate (δ of class 4) in the case of Exemplary Patient C, the method ultimately predicts a much more even probability between very high and very low relapse rates for this patient because the predictions (the fourth column of each confusion matrix in FIGS. 10a-10f) for all of the algorithms misclassify almost as many truly very slowly-relapsing (class 1) patients as very fast (class 4), as compared to correctly classifying very-fast relapsing patients. That is, the upper right corner numbers in each matrix are nearly as large as those in the lower right.

Next, a probability distribution function is selected within each bin (class) to represent the probability distribution of predicted true relapse rate δ within each class for each patient. For example, this function may be assumed to be uniform, normal, log-normal, exponential, or any other function, and may depend on the nature of the response variable. In this example, a uniform probability distribution is selected within each bin (class).

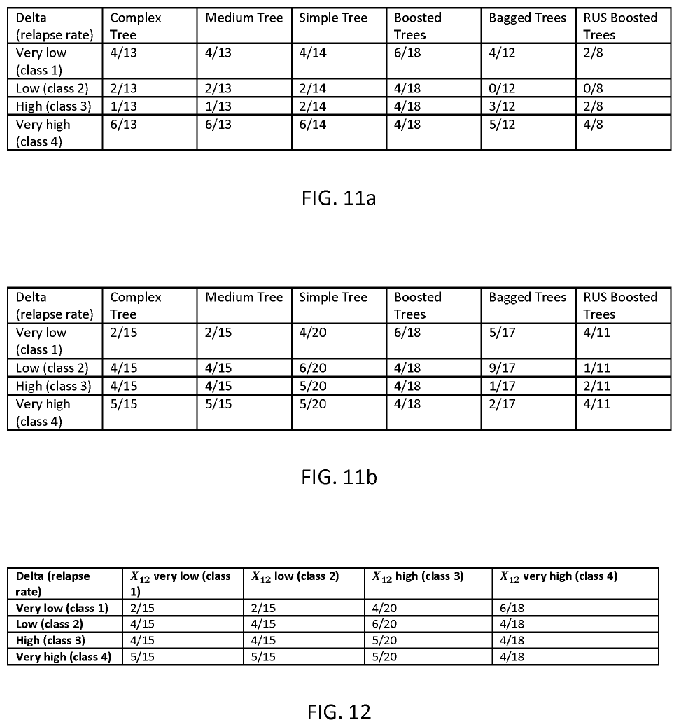

To predict the modeled number of mutants X₁₂* at the start of induction treatment, additional probability distributions are constructed from the training data set for each class of 6. These are shown in FIG. 12 and are valid for both Exemplary Patients C and D, in fact for any patients for which the predictive algorithms are trained on the same training data set of 45 patients. Constructing these probability distributions does not require running additional ML algorithms with X₁₂* as a response variable in this exemplary embodiment. In this example, the possible outcomes for X₁₂* are divided into four classes as follows: X₁₂*<13, 13≤X₁₂*<26, 26≤X₁₂*39, 39≤X₁₂*.

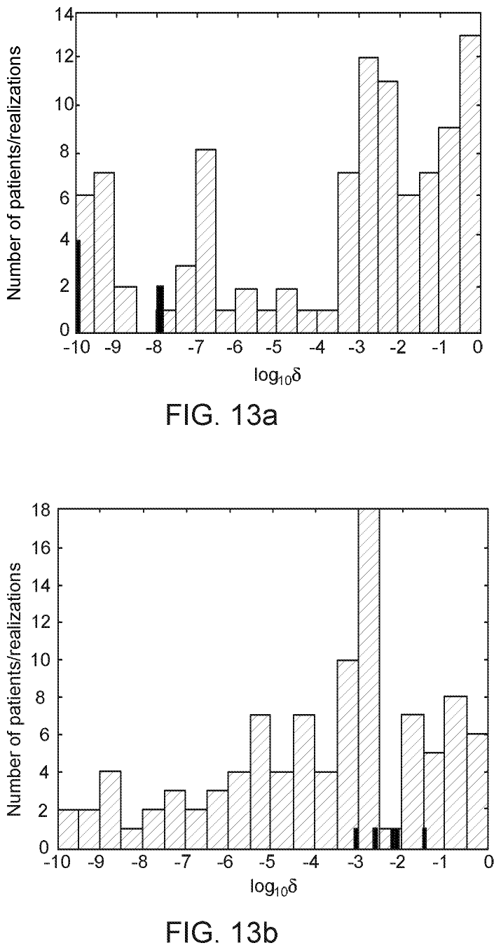

The remaining two parameters of the model for this example are the initial decay rate γ of the cancer cell population (the net kill rate of the induction therapy on the sensitive cell population) and the number of sensitive cancer cells X₀ at the start of induction therapy. These both can be measured at or near the beginning of therapy for each patient and therefore need not be predicted. Multiple probabilistic predictions may then be modeled of the model parameters for Exemplary Patients C and D by randomly sampling the probability distributions shown in FIGS. 11 and 12. The above probability distributions may be randomly sampled, e.g., 100 times for each patient in this example, and the predicted true relapse rate δ for these realizations are given in FIGS. 13a and 13b (cross-hatched histogram), where they are compared to the results from the actual relapse rates for patients of the same classes as Exemplary Patient C and Exemplary Patient D, respectively, within the holdout test data set (solid histogram). FIG. 13a shows Exemplary Patient C and FIG. 13b shows Exemplary Patient D.

Second Exemplary Method Embodiment Step (h)

In this optional Step (h), a mathematical model and the model parameters predicted in Step (g) are used to generate stochastic realizations of the predicted number of cancer cells versus time and their resistance status in the new patient (and hence the patient's treatment outcome) under any proposed treatment for which parameters have been determined in Step (g). Each stochastic realization is an alternative possible future for the evolution of the patient's cancer for Exemplary Patients C and D. In this example we predict the time to progression of the tumor for Exemplary Patients C and D under the same treatment plan and the same mathematical model used on the training data set patients. Alternatively the mathematical model and the model parameters can be used to generate any clinically useful predictions in the new patient for each set of model parameters obtained in step (g) under a new proposed treatment plan that includes at least one treatment employed in step (b) or (d) for which model parameters have been determined in step (g).

Note that while the individual treatments (e.g., drugs or drug combinations) must be chosen from those processed in Step (g) in order to predict the model parameters as responses to the ML algorithms, the timing, duration, and (in this example embodiment) the order of the individual proposed treatments do not have to be chosen until this Step (h). Any arbitrary timing or duration can be proposed for the treatments selected for the treatment plan for Exemplary Patient C and modeled in this example embodiment of Step (h). In this example, the probability versus time of tumor progression is predicted, which is defined as the point in time when the tumor shifts from shrinking to growing, that is, when the resistant cancer cell population begins to exceed the sensitive cancer cell population.

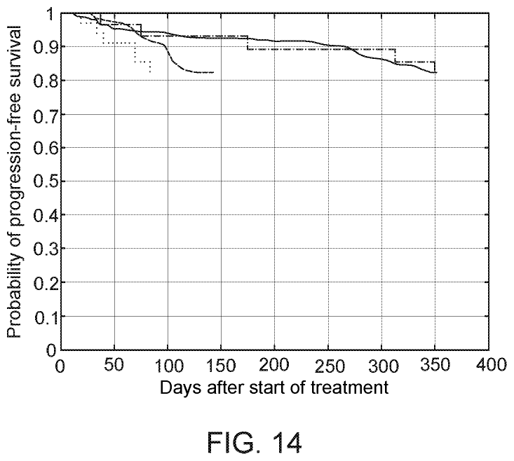

Using the predicted model parameters obtained in Step (g), “synthetic Kaplan-Meier curves” (SKM curves) are produced for both Exemplary Patients C and D as shown in FIG. 14, which shows the SKM curves generated with 100 stochastic realizations for each example patient, compared to Kaplan-Meier curves generated by the actual relapse data for the independent holdout patients of the same predicted classes as the exemplary patients, giving an illustration of the predictive accuracy for this exemplary patient data set under this example embodiment. The SKM curve for Exemplary Patient C is the dashed curve, the SKM curve for Exemplary Patient D is the solid curve, the actual KM curve for Exemplary Patient C is the dotted curve, and the actual KM curve for Exemplary Patient D is the dot-dashed curve.

Second Exemplary Method Embodiment Step (i)

In optional Step (i), Step (f) is continuously or repeatedly performed for the new patient, which may be done before, during, and/or after Steps (g) and (h). This provides updated measurements of tumor size biomarkers for the new patient, which may be used to revise and improve the predictions of the tumor evolution and outcome for the example patients under the proposed treatment plan or any alternative treatment plan in Step (j).

Second Exemplary Method Embodiment Step (j)

In this optional Step (j), in one embodiment the model parameters predicted in Step (g) for the new patient are dynamically revised as more measurements become available from Step (i), with the aim of improving the accuracy and reliability of the predictions of the tumor evolution under the currently selected or any alternative treatment plan. As additional treatments are applied to this patient, their associated model parameters can also be used as additional predictors and used in Step (g) again to predict the model parameters for other future planned treatments.

Second Exemplary Method Embodiment Step (k)

In this optional Step (k), constraints are specified on proposed treatments or on the overall proposed plan. These can be, for example, motivated by trying to limit side-effects, accommodate patient lifestyle, by financial constraints, or for any other reason. Examples include limiting treatment duration for a specific drug to six months or less between 3-month rest periods, specifying a 3-month holiday from all treatment beginning twelve months after treatment start, limiting treatment options to treatments for which data are available from Step (d) exceeding a specified quality or quantity threshold, or excluding a specific drug combination from consideration.

Second Exemplary Method Embodiment Step (l)

In this optional Step (l), the planned treatment protocol is optimized under the constraints imposed in Step (k), or under no constraints, using a mathematical optimization technique with an objective benefitting the patient. Exemplary objectives include maximizing the time before the new patient's cancer reaches a lethal condition, maximizing the time before the patient's cancer begins to progress or grow beyond a certain size, probability of cure, minimization of the number of resistant subpopulations, minimization of the number of treatments that yields no progression in a specified timeframe, maximizing the probability of avoiding progression prior to a planned life event (such as birth of a grandchild), or any other objective that can be derived from the predictions of Step (h) and/or Step (j).

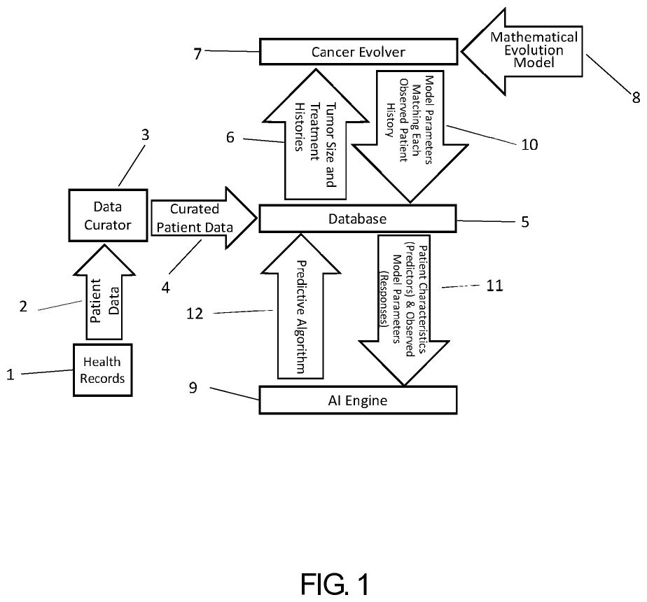

Turning to the drawings, FIG. 1 is a flow chart of an exemplary method for training a system on patient data using system comprising a cancer evolver, an artificial-intelligence (AI) engine, a database, and one or more user interfaces, which may be included in a single electronic device or distributed over multiple devices that communicate with each other over a local and/or wide area network (not shown). The system first identifies a set of health records 1 in the database 5 or elsewhere and extracts desired patient data 2 on desired identified patients by any convenient method. The health records 1 may be stored on paper and/or on electronic media, e.g., which may be managed by a commercially available electronic health records system such as EPIC®.

Optionally, the patient data 2 may be extracted from the health records 1 by a data curator 3, which may be a human, a software program, or both. For example, the optional data curator 3 may select the specific desired data fields and check them for errors and relevance and produce curated data 4 that is then stored in database 5 such that the curated data 4 has been formatted to be compatible for later use and has errors flagged or repaired and has any error-containing or irrelevant data either removed or flagged in the database 5. The system performs these processes for each patient on which the software is being trained. The database 5 may be stored in a single location or multiple locations and may be administered by one or more database management programs such as SQL or MongoDB.

In an exemplary embodiment, the following specific data 6 is then extracted from the database 5: biomarker measurements indicating tumor size or direct measurements of tumor size; treatment start and stop dates; and the drugs, dosages, and administration techniques comprising and uniquely identifying each treatment. These data 6 are fed into a cancer evolver computer program 7 that uses a mathematical model 8 to model the time history of each patient's cancer and all of its relevant subpopulations resistant to the treatments applied to the patient.

The resulting model parameters 10 for each patient are then stored in the database 5. These model parameters 10 may include, for example, the modeled kill rates or strengths of each treatment, the random and/or mutagenic mutation rates or activities associated with each treatment, the untreated growth rates of each patient's cancer, parameters describing the interaction of the cancer with the microenvironment, and/or other parameters. The important requirement is that these parameters collectively are sufficient to predict the evolution of the cancer (i.e., the tumor size versus time) according to the mathematical model, given at least one predicted treatment plan, and preferably any arbitrary treatment plan consisting of the treatments being used on this patient. The above activities are then repeated for each patient that is being used to train the system. This should be as many patients as possible but should be at least ten patients for each treatment or at least one patient for each treatment.

Next, specific patient parameters are identified in the database 5 from the curated patient data 4 and fed into the AI Engine 9 along with the Model Parameters 10 for each of a number of patients. These Patient Parameters may be identified by any method, e.g., are identified using a scientific rationale or demonstrated correlation with the parameters of the mathematical model. For example, it has been observed that the 17p deletion in the myeloma cancer genome is correlated with increased resistance to drugs and earlier relapse, and it is thought this is due to a loss of gene repair mechanisms resulting from the mutation. Thus, a correlation between the presence of this mutation and an increased mutation rate into a drug resistant phenotype in the mathematical model may be expected. The AI Engine 9 then seeks a predictive relationship between the selected patient parameters as predictors and the observed model parameters as response (collectively 11), and it generates a Predictive Algorithm 12 relating these predictors and responses.

To accomplish this, the AI Engine 9 may employ ML algorithms such as linear regression, support vector machines, trees, ensembles, or any other algorithms, or it may use some other artificial intelligence methodology such as neural networks or deep learning. In contrast to the Cancer Evolver 7, the AI Engine 9 is not required to use models based on biological or physical science or past observations (other than those provided as inputs 11) to create the Predictive Algorithm 9. The features and parameters describing the Predictive Algorithm 12 are then stored in the Database 5 for future use.

FIG. 2 shows an example of a modeling plot of the log-base10 of the tumor size versus time for the myeloma cancer of Exemplary Patient A. The data points are tumor size measurements derived from blood biomarkers, the shaded regions indicate treatment periods, and the lines are modeled tumor size curves generated by the cancer evolver. For this patient, three drugs were used at various dose levels: lenalidomide (Revlimid®), bortezomib (Velcade®), and dexamethasone. Treatment was initiated on Day 1 with a 21-day cycle of RVD treatment consisting of 25 mg/day of lenalidomide on days 1-14, 1.3 mg/m² of bortezomib on days 1,4,8,11, and 20 mg/day of dexamethasone on days 1,2,4,5,8,9,11,12. This was followed by RDLiteV treatment, which was the same s RVD except with the bortezomib dose reduced to 1 mg/m², followed by a break in treatment, followed by RLiteVD, which was the same as RDLiteV except the dexamethasone dose was reduced to 10 mg/day, followed by RevMaint10 maintenance therapy with 10 mg/day of lenalidomide continuously (except for a break between days 250 and 275, followed by RevMaint5, which is 5 mg/day of lenalidomide continuously.

Note that resistance to RevMaint5 becomes apparent around Day 350 as the myeloma biomarker starts to increase at that time. The biomarker used here is the concentration of serum kappa free light chains secreted by the myeloma. The error bars represent the 30% typical uncertainty in these measurements due to clinical and laboratory variability. The number of tumor cells is determined by calibrating free kappa light chain measurements against absolute measurements made by bone marrow next-generation sequencing (NGS) on a number of patients. It can therefore be considered an estimate accurate to within a factor of ten or better.

High accuracy of this number on an absolute basis is not required to achieve utility of the systems and methods herein, however, because, among other reasons, similar scaling factors are used for all patients and so the relative accuracy across time, across various subpopulations, and across patients is not subject to this uncertainty. The solid line is the modeled total cancer cell population, the dotted line is the modeled population of cancer cells sensitive to all the applied treatments, and the dashed line is the modeled population resistant to lenalidomide alone but sensitive to the combination of all three drugs. In order to fit the observed biomarkers indicating tumor size versus time, the model must assume certain kill rates to match the slopes of the data during each treatment, and mutation rates to match the timing of the emergence of resistance to lenalidomide as observed.

For this Exemplary Patient A, the kill rates and mutation rates inferred by the model are given in the previous section. The only relevant mutation rate inferred by the model is the rate of mutation of fully sensitive cancer cells into cells resistant to lenalidomide alone (RMaint10 and RMaint5). Note this results in the first resistant mutant being born soon after initiation of the cancer and well before diagnosis or start of treatment according to the model of this example.

FIG. 3 is a plot of predicted mutation rates versus actual modeled mutation rates resulting from modeling twelve patients undergoing lenalidomide single-agent maintenance therapy in addition to other treatments. The predicted mutation rates are generated by feeding many patient parameters (predictors) into competing ML algorithms to determine a relationship between the predictors and the mutation rate (response) of each patient. For this example, the predictors were the RVD kill rate plus the following characteristics of the myeloma cells' chromosomal abnormalities: the presence of the 17p deletion, the presence of the (11;14) translocation, and the overall number of chromosomal deletions or trisomies. Many other possible selections and combinations of predictors are possible.

The data were fed into the MATLAB® Regression Learner application and 19 ML algorithms were run in competition using 5-fold cross validation and their predicted responses (the mutation rates) were compared to the actual mutation rates inferred by the Cancer Evolver and used to train the algorithm(s). In this example, the best results were obtained with a squared exponential Gaussian process regression algorithm. The results shown in FIG. 3 show a match between the logarithms of algorithm's predicted mutation rates for each patient and the actual observed (input) mutation rates with a coefficient of determination R²=0.84 and a RMSE of 0.94.

FIG. 4 is similar to FIG. 3 except that it applies to the kill rates of RVD. In this example, the input predictors were the patient's age, gender, height, weight, BSA, BMI, measured creatinine, peak serum light chain concentration, and calculated glomerular filtration rate. The RMSE=0.02 and R²=0.27 with n=12 patients. Many other selections and combinations of predictors are possible.

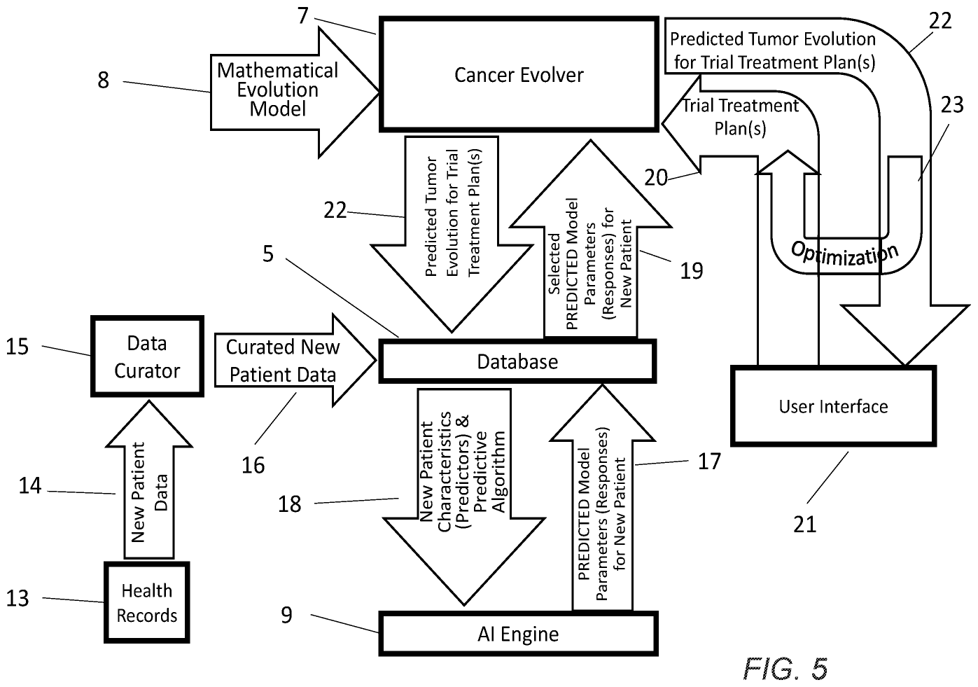

FIG. 5 is a flow chart of an exemplary method for using the software to predict outcomes for various treatment protocols and to optimize such protocols for specific patients using a cancer evolver, an artificial-intelligence (AI) engine, a database, and one or more user interfaces. A new set of health records 13 is accessed by an optional data curator 15 and data for a new patient 14 is curated 16 and stored in the database 5. Optionally, the data curator 15 may be the same as the data curator 3 in FIG. 1. The same Patient Predictors used to train the software in FIG. 1 and the same Predictive Algorithm 12 shown in FIG. 1 are then fed into the AI Engine 9 from the database 5 as 18 and used by the AI Engine 9 to generate the Predicted Model Parameters 17 for this new patient and these are stored in the database 5.

With continued reference to FIG. 5, a user then engages the Cancer Evolver 7 via a user interface 21. The user enters Trial Treatment Plans 20 (proposed treatments and start and end dates for each proposed treatment) via the User Interface 21, which feeds these into the Cancer Evolver 7. The Cancer Evolver 7 then uses the Predicted Model Parameters for the New Patient 19 to predict the evolution of the cancer for this new patient and feeds this information 22 to the Database 9 and back to the user via the User Interface 21. Optionally, trial treatment plans may be stored in the Database 9, and the Cancer Evolver 7 may engage itself automatically to perform this step once it determines sufficient information is stored in the Database 9.

As an optional further step, the user can request the Cancer Evolver 7 to optimize 23 the predicted tumor evolution 22 to maximize patient survival or meet some other objective via a feedback loop 23. The optimization objective can be for example expected progression-free survival, probability of cure, time before exceeding a certain tumor size, minimization of the number of resistant subpopulations, minimization of the number of treatments to yield no progression in a specified timeframe, or any other objective that can be derived from the predicted evolution of the number of tumor cells and its subpopulations with various resistance states relative to specified treatments. The optimization can be performed under no constraints other than limitations imposed by data availability, or under various imposed constraints as desired by the user, including for example, a limit of five allowed treatments, exclusion of certain treatments such as autologous transplant, imposition of a treatment holiday of three months beginning six months in the future, or any other set of constraints that would affect the way the Cancer Evolver 7 is able to utilize the various candidate treatments that might be available.

FIG. 6a is a prediction plot of the log-base10 of the tumor size versus time for the myeloma cancer of Exemplary Patient B under a proposed treatment plan. The lines are tumor size curves predicted by the cancer evolver and the shaded regions indicate treatment periods. For this patient, four drugs are used at various dose levels—lenalidomide (Revlimid®), bortezomib (Velcade®), melphalan (with stem cell transplant), and dexamethasone, comprising five different treatments. The planned treatment is initiated on Day 1 with RVD through Day 56 followed by a short treatment break and then the melphalan/transplant on Day 92, followed by RVLiteD on Days 196-258, followed by RevMaint10 through Day 359, followed by RevMaint5 through Day 752. Note that resistance to RevMaint5 is predicted to emerge around Day 650.

FIG. 6b shows the results of applying the proposed treatment plan from FIG. 6a. The data points are tumor size measurements derived from blood biomarkers. Note that resistance to RevMaint5 becomes apparent around Day 650 as the myeloma biomarker starts to increase rapidly at that time. The biomarker used here is the concentration of serum kappa free light chains secreted by the myeloma. Error bars and other notation are the same as for FIG. 2.

FIGS. 7a-7d show an exemplary model of the cell cycle prior to treatment. This is the cell cycle assumed for the example mathematical model used in Steps 3, 8, and 12 of the first exemplary embodiment of the method. Each cycle starts simultaneously for all cells immediately after cell division. Unmutated cancer stem cells may mutate to become resistant with probability pₘᵤₜₐₜₑ, divide with probability p_{divide}, or differentiate with probability p_{diff}. Differentiation and natural cell death occur at the end of the G1 phase, mutation occurs in the S, G2, or M phases, and division occurs at the end of the cycle. Cross-hatching represents processes with probability less than one.

FIG. 7a shows the cycle for unmutated cancer stem cells, which may differentiate with probability p_{diff} at the end of the G1 phase or may mutate throughout the remainder of the cycle with probability pₘᵤₜₐₜₑ, and may divide with probability p_{divide} at the end of the cycle. FIG. 7b shows unmutated differentiated cells, which perform their non-resistant metabolic roles until natural death, which has a probability p_{die} per cycle. FIG. 7c illustrates mutated stem cells, which retain their ability to divide and mutate. They may differentiate with probability p_{diff} at the end of the G1 phase or may mutate throughout the remainder of the cycle with probability pₘᵤₜₐₜₑ, and may divide with probability p_{divide} at the end of the cycle. The cycle for mutated differentiated cells is shown in FIG. 7d. These cells are resistant but cannot divide or mutate and die off with a probability of p_{die} per cycle.

It is assumed in the first exemplary model that mutation occurs during DNA replication or in other steps leading up to cell division, which implies a cell must divide in order to mutate. So the division probability is implicitly “built in” to the mutation probability. In this exemplary model, p_{divide} is the same for all cells. Because all probabilities are defined per cell cycle, changing p_{divide} without changing pₘᵤₜₐₜₑ changes the probability of mutation per cell division, but not per cell cycle.

FIGS. 8a and 8b show exemplary drug kill models during treatment. Drug kill with probability (a) pₖᵢₗₗ for fully sensitive cells (FIG. 8a) or (b) pₖᵢₗₗ for partially resistant (partially sensitive) cells (FIG. 8b) can occur anywhere throughout the cycle and are overlaid on the processes shown in FIG. 7. For this example, the following assumptions are used by the mathematical model. All cells in a given subpopulation are assumed to have the same probability of kill by a given treatment, but each treatment may have a different kill probability. For each treatment, partially resistant cells may have any kill probability between zero and the full kill probability of sensitive cells.

FIGS. 9a and 9b show mutation diagrams corresponding to (a) two treatments and (b) four treatments, respectively. Each R node corresponds to a phenotype (pool of mutant genotypes) resistant to the treatment specified by the R-subscripts. For example, R₁₃₄ is resistant to treatments 1, 3, and 4 but not 2. The R₀ node is the fully sensitive phenotype. Solid arrows indicate direct mutation and dashed arrows cross-mutation. The nodes represent only their respective cancer stem cell compartments, as the differentiated cancer cells do not divide or mutate. In FIG. 9a, the mutation probabilities are shown for each arrow; they are omitted for clarity in FIG. 9b, which shows the rapid increase in complexity as the number of treatments is increased.

FIG. 9a shows the case of two treatments applied to a tumor stem cell subpopulation R₀^{S} that is sensitive to both treatments but may or may not be resistant to any other treatments. The subpopulations R₁^{S}, R₂^{S}, and R₁₂^{S} are fully resistant to treatment(s) 1, 2 and both 1 and 2, respectively. The mutation rates are shown above each arrow, with pₘ₁ and pₘ₂ the direct mutation rates for resistant subpopulations R₁^{S} and R₂^{S}, respectively, and pₘ₁₂ the cross-mutation rate for the cross-resistant subpopulation R₁₂^{S}.

FIG. 9b shows the case of four treatments and illustrates how the number of possible subpopulations and mutation events grows rapidly with increasing number of treatments. N_{subpop}=2^{N}, N is the number of treatments, and N_{subpop} is the number of possible subpopulations.

So with two treatments, four possible stem subpopulations and three possible mutation processes are tracked. With four treatments this potentially grows to 16 subpopulations and 93 mutation processes, and for seven treatments this potentially becomes 128 subpopulations and 6476 mutation processes. These numbers can potentially be significantly reduced algorithmically, but in a practical clinical situation tracking large numbers of possible mutant subpopulations and possible mutation processes may still be required if many different treatments are being employed.

FIGS. 10a-10f show a set of six confusion matrices showing the predicted versus actual relapse rates δ for six decision-tree machine learning algorithms. These decision-tree algorithms use the following approaches: complex tree (maximum of 100 splits), medium tree (maximum 20 splits), simple tree (maximum 4 splits), boosted tree ensemble (AdaBoost with 30 learners), bagged tree ensemble (30 learners), and randomly under-sampled boosted tree ensemble (30 learners). The ML algorithm response used in this case was log₁₀ δ, the log₁₀ of the rate of progression in the model.

FIGS. 11a and 11b are tables of the probability distributions of the predicted true relapse rate δ for Exemplary Patient C in FIG. 11a and for Exemplary Patient D in FIG. 11b. These tables are generated by the voting of the six decision-tree machine learning algorithms that generated the confusion matrices of FIGS. 10a-10f using the uniform probabilities for each algorithm selected for this exemplary embodiment.

FIG. 12 shows predicted number of mutants X₁₂* at the start of induction treatment based on the model. These are constructed from additional probability distributions from the training data set for each class of δ. These are valid for both Exemplary Patients C and D, in fact for any patients for which the predictive algorithms are trained on the same training data set of 45 patients.

FIGS. 13a and 13b show histograms of modeled multiple probabilistic predictions of the model parameters for Exemplary Patients C and D by randomly sampling the probability distributions given in FIGS. 11 and 12. These probability distributions were sampled 100 times for each patient in this example. The predicted relapse rates for these realizations are shown as the cross-hatched histograms, and they are compared to the results from the actual relapse rates for patients of the same class within the holdout test data set (solid histograms). FIG. 13a shows Exemplary Patient C and FIG. 13b shows Exemplary Patient D.

The number of patients shown in the histogram of actual relapse rates is limited by the number of patients in the holdout data-set, while the number of realizations shown in the histogram of predicted relapse rates is limited only by the number of realizations run computationally.

FIG. 14 shows “synthetic Kaplan-Meier curves” (SKM curves) for both Exemplary Patients C and D generated from the predicted model parameters obtained in Step (g) of the second exemplary embodiment described above. This shows SKM curves generated with 100 stochastic realizations for each example patient, compared to Kaplan-Meier (KM) curves generated by the actual relapse data for the independent holdout patients of the same predicted classes as the example patients, giving an illustration of the predictive accuracy for this example patient data set under this exemplary embodiment.

The SKM curve for Exemplary Patient C is the dashed curve, the SKM curve for Exemplary Patient D is the solid curve, the actual KM curve for Exemplary Patient C is the dotted curve, and the actual KM curve for Exemplary Patient D is the dot-dashed curve.

In describing representative embodiments, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

I claim:

1. A method for predicting the outcome of treatment protocols in hematological cancer patients, the method comprising a training phase and a utilization phase, together comprising the steps of:
(a) collecting data relating to the characteristics of a patient and the patient's cancer;
(b) determining a treatment and cancer history of the patient, including measurements at three or more times from which the number and growth rate of the number of cancer cells in the patient can be at least approximately inferred without imaging the bulk tumor;
(c) using a mathematical model and results of step (b) to infer model parameters, including at least one kill rate and one mutation rate, associated with the response of the patient's cancer to at least one treatment in the patient's treatment history;
(d) repeating steps (a)-(c) for multiple patients;
(e) employing machine-learning algorithms to search for mathematical relationships between the characteristics collected in steps (a) and (d) and the model parameters inferred in steps (c) and (d);
(f) collecting data relating to characteristics of a new patient and the new patient's cancer;
(g) using the new patient data and the machine-learning algorithms to predict at least one model parameter or mathematical function thereof for the new patient, including at least one kill rate and one mutation rate; and
(h) using a mathematical model and at least one of the model parameters predicted for the new patient to predict one or both of a number of cancer cells versus time and a resistance status of the cancer cells in the new patient under a new proposed treatment plan that includes at least one treatment employed in step (b) or (d) for which parameters have been determined in step (g).

2. The method of claim 1, comprising the additional steps of:
(i) continuously or repeatedly performing step (f) for the new patient, and
(j) repeating steps (a) through (e) and steps (g) and (h) using the results of step (i).

3. The method of claim 1, wherein the new proposed treatment plan is optimized for a desired objective benefitting the new patient.

4. The method of claim 3, wherein the new treatment plan is optimized using the additional steps of:
(k) optionally specifying one or more constraints on proposed treatments or on the overall proposed treatment plan; and
(l) optimizing the new proposed treatment plan using a mathematical optimization technique with an objective benefitting the new patient.

5. The method of claim 1 wherein the mathematical model of step (c) predicts a time to produce a first mutant resistant to at least one treatment by comparing a cumulative probability of producing the first mutant to a threshold value.

6. The method of claim 1, wherein the mathematical model predicts growth, death, and mutation rates of cancer cells over time.

7. The method of claim 1, wherein the patient, multiple patients, and new patient are each a myeloma patient.

8. The method of claim 1, wherein the patient, multiple patients, and new patient are each a hematological cancer patient.

9. The method of claim 1, wherein the tumor-size measurements made in steps (b) and (d) are made from a liquid biopsy.

10. A method for predicting the outcome of treatment protocols in cancer patients, the method comprising a training phase and a utilization phase, together comprising the steps of:
(a) collecting data relating to characteristics of a patient and the patient's cancer;
(b) determining a treatment and cancer history of the patient, including measurements at three or more times from which the size of at least one tumor or the number of cancer cells in the patient can be at least approximately inferred;
(c) using a mathematical model and results of step (b) to infer model parameters associated with the response of the patient's cancer to at least one treatment in the patient's treatment history;
(d) repeating steps (a)-(c) for multiple patients;
(e) employing one or more machine-learning algorithms to classify one or more of the model parameters, or a mathematical function thereof, based on mathematical relationships between the characteristics collected in steps (a) and (d) and the model parameters inferred in steps (c) and (d);
(f) collecting data relating to characteristics of a new patient and the new patient's cancer; and
(g) constructing one or more binned probability distributions of at least one model parameter predicted in step (e) or mathematical function thereof for the new patient with one bin for each possible class of each model parameter, randomly sampling one or more of the one or more probability distributions to determine a bin for the model parameter or mathematical function thereof for the new patient and constructing and randomly sampling a separate probability distribution within the bin to determine a value for each model parameter or mathematical function thereof.

11. The method of claim 10, comprising the additional step of:
(h) using a mathematical model to generate one or more stochastic realizations of a number of cancer cells versus time and a resistance status of the cancer cells in the new patient for each set of model parameters obtained in step (g) under a new proposed treatment plan that includes at least one treatment employed in step (b) or (d) for which model parameters have been determined in step (g).

12. The method of claim 10, comprising the additional steps of:
(i) continuously or repeatedly performing step (f) for the new patient, and
(j) repeating steps (a) through (e) and steps (g) and (h) using the results of step (i).

13. The method of claim 10, wherein the new proposed treatment plan is optimized for a desired objective benefitting the new patient.

14. The method of claim 10, wherein the new treatment plan is optimized using the additional steps of:
(k) optionally specifying one or more constraints on proposed treatments or on the overall proposed treatment plan; and
(l) optimizing the new proposed treatment plan using a mathematical optimization technique with an objective benefitting the new patient.

15. The method of claim 10, wherein more than one machine learning algorithm is used in step (g) and a binned probability distribution of at least one model parameter predicted in step (e) or mathematical function thereof is constructed for each algorithm for the new patient with one bin for each possible class of each model parameter or mathematical function thereof, weighting each binned probability distribution, randomly sampling one or more of the weighted probability distributions to determine the bin for the model parameter or mathematical function thereof for the new patient and constructing and randomly sampling a separate probability distribution within the bin to determine a value for each model parameter or mathematical function thereof.

16. A system for implementing the method of claim 1 that comprises computer hardware and software that further comprises a cancer evolver, an artificial-intelligence engine, a database, and a user interface.

17. A system for predicting the outcome of treatment protocols in hematological cancer patients, comprising:
a cancer evolver configured to:
(a) determine a treatment and cancer history of a patient, including measurements at three or more times from which the number and growth rate of the number of cancer cells in the patient can be at least approximately inferred without imaging the bulk tumor;
(b) use a mathematical model and results of step (a) to infer model parameters, including at least one kill rate and one mutation rate, associated with the response of the patient's cancer to at least one treatment in the patient's treatment history; and
(c) repeat steps (a)-(b) for multiple patients; and
an artificial-intelligence engine configured to:
(d) collect data relating to the characteristics of a patient and the patient's cancer;
(e) repeat step (d) for multiple patients;
(f) employ machine-learning algorithms to search for mathematical relationships between the characteristics collected in steps (d) and (e) and the model parameters inferred in steps (b) and (c); and
(g) use data collected relating to characteristics of a new patient and the new patients cancer and the machine-learning algorithms to predict at least one model parameter or mathematical function thereof for the new patient, including at least one kill rate and one mutation rate
the cancer evolver further configured to:
(h) use a mathematical model and at least one of the model parameters predicted for a new patient to predict one or both of a number of cancer cells versus time and a resistance status of the cancer cells in the new patient under a new proposed treatment plan that includes at least one treatment employed in step (b) or (c) for which parameters have been determined in step (g).

18. The system of claim 17, wherein the artificial-intelligence engine is configured to optimize the new treatment by:
(h) optionally specifying one or more constraints on proposed treatments or on the overall proposed treatment plan; and
(i) optimizing the new proposed treatment plan using a mathematical optimization technique with an objective benefitting the new patient.

19. The system of claim 17, wherein the cancer evolver is configured to use the mathematical model of step (c) to predict a time to produce a first mutant resistant to at least one treatment by comparing cumulative probability of producing the first mutant to a threshold value.

20. The system of claim 17, further comprising a database communicating with the cancer evolver, the cancer evolver configured to collect the characteristics from the database.

21. A method for treating a cancer patient utilizing a prediction of outcomes of treatment protocols in other cancer patients, the method comprising the steps of:
(a) collecting data relating to characteristics of a patient and the patient's cancer;
(b) determining a treatment and cancer history of the patient, including measurements at three or more times from which a size of at least one tumor or a number of cancer cells in the patient can be at least approximately inferred;
(c) using a mathematical model and the results of step (b) to infer model parameters associated with the response of the patient's cancer to at least one treatment in the patient's treatment history;
(d) repeating steps (a)-(c) for multiple patients;
(e) employing machine-learning algorithms to search for mathematical relationships between the characteristics collected in steps (a) and (d) and the model parameters inferred in steps (c) and (d);
(f) collecting data relating to characteristics of a new patient and the new patient's cancer;
(g) using the new patient data and the machine-learning algorithms to predict at least one model parameter or mathematical function thereof for the new patient; and
(h) using a mathematical model and at least one of the model parameters predicted for the new patient to predict one or both of a number of cancer cells versus time and a resistance status of the cancer cells in the new patient under a new proposed treatment plan that includes at least one treatment employed in step (b) or (d) for which parameters have been determined in step (g); and
(i) treating the new patient based at least in part on the results of step (h).

22. The method of claim 21, comprising the additional steps of:
(j) continuously or repeatedly performing step (f) for the new patient, and
(k) repeating steps (a) through (e) and steps (g) and (h) using the results of step (j).

23. The method of claim 21, wherein the new proposed treatment plan is optimized for a desired objective benefitting the new patient.

24. The method of claim 23, wherein the new treatment plan is optimized using the additional steps of:
(l) optionally specifying one or more constraints on proposed treatments or on the overall proposed treatment plan; and
(m) optimizing the new proposed treatment plan using a mathematical optimization technique with an objective benefitting the new patient.

25. The method of claim 21, wherein the mathematical model of step (c) predicts a time to produce a first mutant resistant to at least one treatment by comparing cumulative probability of producing the first mutant to a threshold value.

26. The method of claim 21, wherein the mathematical model predicts growth, death, and mutation of cancer cells over time.

27. The method of claim 21, wherein the patient, multiple patients, and new patient are each a myeloma patient.

28. The method of claim 21, wherein the patient, multiple patients, and new patient are each a hematological cancer patient.

29. The method of claim 21, wherein the tumor-size measurements made in steps (b) and (d) are made from a liquid biopsy.

30. A method for treating cancer patients utilizing a prediction of outcomes of treatment protocols in other cancer patients, the method comprising the steps of:
(a) collecting data relating to characteristics of a patient and the patient's cancer;
(b) determining treatment and cancer history of the patient, including measurements at three or more times from which a size of at least one tumor or a number of cancer cells in the patient can be at least approximately inferred;
(c) using a mathematical model and results of step (b) to infer model parameters associated with the response of the patient's cancer to at least one treatment in the patient's treatment history;
(d) repeating steps (a)-(c) for multiple patients;
(e) employing one or more machine-learning algorithms to classify one or more of the model parameters, or a mathematical function thereof, based on mathematical relationships between the characteristics collected in steps (a) and (d) and the model parameters inferred in steps (c) and (d);
(f) collecting data relating to characteristics of a new patient and the new patient's cancer; and
(g) constructing one or more binned probability distributions of at least one model parameter predicted in step (e) or mathematical function thereof for the new patient under a new proposed treatment plan that includes at least one treatment employed in step (b) or (d), with one bin for each possible class of each model parameter, randomly sampling one or more of one or more probability distributions to determine a bin for the model parameter or mathematical function thereof for the new patient and constructing and randomly sampling a separate probability distribution within the bin to predict a value for each model parameter or mathematical function thereof; and
(h) treating the new patient based at least in part on the results of step (g).

31. The method of claim 30, comprising the additional step of:
(i) using a mathematical model to generate one or more stochastic realizations of a number of cancer cells versus time and a resistance status of the cancer cells in the new patient for each set of model parameters obtained in step (g) under a new proposed treatment plan that includes at least one treatment employed in step (b) or (d) for which model parameters have been determined in step (g).

32. The method of claim 30, comprising the additional steps of:
(j) continuously or repeatedly performing step (f) for the new patient, and
(k) repeating steps (a) through (e) and steps (g) and (h) using the results of step (k).

33. The method of claim 30, wherein the new proposed treatment plan is optimized for a desired objective benefitting the new patient.

34. The method of claim 33, wherein the new treatment plan is optimized using the additional steps of:
(l) specifying one or more constraints on proposed treatments or on the overall proposed treatment plan; and
(m) optimizing the new proposed treatment plan under constraints using a mathematical optimization technique with an objective benefitting the new patient.

35. The method of claim 21, wherein treating the new patient comprises:
preparing one or more agents for administration to the new patent according to the at least one treatment; and administering the one or more agents to the new patient.

36. A method for treating a cancer patient, comprising:
identifying a treatment plan for the cancer patient using the method of claim 1; and
administering one or more agents included in the identified treatment plan to the cancer patient.

37. The method of claim 10, comprising the additional step of:
(h) using a mathematical model to generate one or more clinically useful predictions in the new patient for each set of model parameters obtained in step (g) under a new proposed treatment plan that includes at least one treatment employed in step (b) or (d) for which model parameters have been determined in step (g).

38. The method of claim 10, comprising the additional step of:
(h) using a mathematical model to generate one or more synthetic Kaplan-Meier curves in the new patient for each set of model parameters obtained in step (g) under a new proposed treatment plan that includes at least one treatment employed in step (b) or (d) for which model parameters have been determined in step (g).
